SEQUENCE LISTING (36 sequences)

SEQ ID NO: 1 - DNA, 1620 bp, Mus musculus
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagatcccatgaaggagatcctccccatgtcatggctcagataccttctgctggtccttggttgtttttctggcttcttctcctgcgtctacattttggtcaagtgccggtaccttgggccatggctgaagacagttctcaagtgccacatcccagatccttctgagttcttctcccagctgagctcccagcatgggggagaccttcagaaatggctctcctcgcctgtccccttgtccttcttcagccccagtggccctgcccctgagatctctccgctggaagtgctcgacggagattccaaggccgtgcagctgctcctgttacagaaggactctgcccctttaccctcgcccagcggccactcacaggccagctgcttcaccaaccagggctacttcttcttccatctgcccaatgccttggagatcgaatcctgccaggtgtacttcacctatgacccctgtgtggaagaggaggtggaggaggatgggtcaaggctgcccgagggatctccccacccacctctgctgcctctggctggagaacaggatgactactgtgccttcccgcccagggatgacctgctgctcttctccccgagcctcagcacccccaacactgcctatgggggcagcagagcccctgaagaaagatctccactctccctgcatgagggacttccctccctagcatcccgtgacctgatgggcttacagcgccctctggagcggatgccggaaggtgatggagaggggctgtctgccaatagctctggggagcaggccagtgtcccagaaggcaaccttcatgggcaagatcaggacagaggccagggccccatcctgaccctgaacaccgatgcctatctgtctcttcaagaactacaggcccaagattcagtccacctaatatga

SEQ ID NO: 2 - protein, 539 aa, Mus musculus
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 3 - DNA, 1389 bp, Artificial sequence
  Synthetic Sequence
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaaagaatcaaggaggatgggatcctgtcttgccaagtgtcaccattctgagtttgttctctgtgtttttgttggtcatcttagcccatgtgctatggaaaaaaaggattaaacctgtcgtatggcctagtctccccgatcataagaaaactctggaacaactatgtaagaagccaaaaacgagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatga

SEQ ID NO: 4 - protein, 462 aa, Artificial sequence
  Synthetic Sequence
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAKNQGGWDPVLPSVTILSLFSVFLLVILAHVLWKKRIKPVVWPSLPDHKKTLEQLCKKPKTSLNVSFNPESFLDCQIHEVKGVEARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 5 - DNA, 1608 bp, Mus musculus
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcagatcccatgaaggagatcctccccatgtcatggctcagataccttctgctggtccttggttgtttttctggcttcttctcctgcgtctacattttggtcaagtgccggtaccttgggccatggctgaagacagttctcaagtgccacatcccagatccttctgagttcttctcccagctgagctcccagcatgggggagaccttcagaaatggctctcctcgcctgtccccttgtccttcttcagccccagtggccctgcccctgagatctctccgctggaagtgctcgacggagattccaaggccgtgcagctgctcctgttacagaaggactctgcccctttaccctcgcccagcggccactcacaggccagctgcttcaccaaccagggctacttcttcttccatctgcccaatgccttggagatcgaatcctgccaggtgtacttcacctatgacccctgtgtggaagaggaggtggaggaggatgggtcaaggctgcccgagggatctccccacccacctctgctgcctctggctggagaacaggatgactactgtgccttcccgcccagggatgacctgctgctcttctccccgagcctcagcacccccaacactgcctatgggggcagcagagcccctgaagaaagatctccactctccctgcatgagggacttccctccctagcatcccgtgacctgatgggcttacagcgccctctggagcggatgccggaaggtgatggagaggggctgtctgccaatagctctggggagcaggccagtgtcccagaaggcaaccttcatgggcaagatcaggacagaggccagggccccatcctgaccctgaatcaagaagaagcgtatgtcaccatgtctagtttttaccaaaacaaatga

SEQ ID NO: 6 - protein, 535 aa, Mus musculus
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNQEEAYVTMSSFYQNK

SEQ ID NO: 7 - DNA, 1344 bp, Mus musculus
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcacagaggagacagggcctcctggtcccacgctggcaatggtcagccagcatccttgtagttgtgcccatctttcttctgctgactggctttgtccaccttctgttcaagctgtcacccaggctgaagagaatcttttaccagaacattccatctcccgaggcgttcttccatcctctctacagtgtgtaccatggggacttccagagttggacaggggcccgcagagccggaccacaagcaagacagaatggtgtcagtacttcatcagcaggctcagagtccagcatctgggaggccgtcgccacactcacctatagcccggcatgccctgtgcagtttgcctgcctgaagtgggaggccacagccccgggcttcccagggctcccaggctcagagcatgtgctgccggcagggtgtctggagttggaaggacagccatctgcctacctgccccaggaggactgggccccactgggctctgccaggccccctcctccagactcagacagcggcagcagcgactattgcatgttggactgctgtgaggaatgccacctctcagccttcccaggacacaccgagagtcctgagctcacgctagctcagcctgtggcccttcctgtgtccagcagggcctga

SEQ ID NO: 8 - protein, 447 aa, Artificial sequence
  Synthetic Sequence
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAQRRQGLLVPRWQWSASILVVVPIFLLLTGFVHLLFKLSPRLKRIFYQNIPSPEAFFHPLYSVYHGDFQSWTGARRAGPQARQNGVSTSSAGSESSIWEAVATLTYSPACPVQFACLKWEATAPGFPGLPGSEHVLPAGCLELEGQPSAYLPQEDWAPLGSARPPPPDSDSGSSDYCMLDCCEECHLSAFPGHTESPELTLAQPVALPVSSRA

SEQ ID NO: 9 - DNA, 1614 bp, Artificial sequence
  Synthetic Sequence
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaagacctgctggcgaacctgaagctggatgggaccctcatatgttgctgctgctggccgtgctgatcatcgtgctggtgttcatgggcctgaagatccatctgccttggagactgtggaagaaaatctgggcccctgtgcctactcctgagagcttcttccagccactgtacagagagcacagcggcaacttcaagaaatgggtcaacacccctttcaccgccagcagtatcgagctggtgcctcagagcagcaccacaacatctgccctgcacctgtctctgtaccccgccaaagagaagaagttccctggcctgcctggactggaagaacagctggaatgtgacggcatgagcgagcctggccactggtgtatcattcctctggctgctggacaggccgtgtccgcctatagcgaggaaagagacagaccctacggcctggtgtccatcgacacagtgacagtgggagatgccgagggcctgtgtgtgtggccttgtagctgtgaagatgacggctaccctgccatgaacctggatgccggaagagagagcggccctaactctgaggatctgctgctcgtgaccgatcctgccttcctgtcttgcggctgtgtgtctggatctggcctgagactcggaggctctcctggaagcctgctggatagactgagactgagcttcgccaaagaaggcgactggaccgccgatcctacttggagaacaggatctcctggcggcggaagcgaatctgaagcaggttctccacctggcctggacatggacacattcgactctggcttcgccggcagcgattgtggaagccctgtggaaacagacgagggcccacctagaagctacctgagacagtgggtcgtgcggacacctcctccagttgattctggcgcccagtcctcttga

SEQ ID NO: 10 - protein, 537 aa, Artificial sequence
  Synthetic Sequence
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPAGEPEAGWDPHMLLLLAVLIIVLVFMGLKIHLPWRLWKKIWAPVPTPESFFQPLYREHSGNFKKWVNTPFTASSIELVPQSSTTTSALHLSLYPAKEKKFPGLPGLEEQLECDGMSEPGHWCIIPLAAGQAVSAYSEERDRPYGLVSIDTVTVGDAEGLCVWPCSCEDDGYPAMNLDAGRESGPNSEDLLLVTDPAFLSCGCVSGSGLRLGGSPGSLLDRLRLSFAKEGDWTADPTWRTGSPGGGSESEAGSPPGLDMDTFDSGFAGSDCGSPVETDEGPPRSYLRQWVVRTPPPVDSGAQSS

SEQ ID NO: 11 - DNA, 1497 bp, Mus musculus
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaagcgatctggaccctctgatcctgacactgagcctgatcctggtgctgatctccctgctgctgacagtgctggccctgctgagccacagaagaaccctgcagcagaagatctggcctggcatcccatctccagagagcgagttcgagggcctgttcaccacacacaagggcaacttccagctgtggctgctgcagcgagatggctgtctttggtggtcccctggcagcagctttcctgaggatccaccagctcacctggaagtgctgagcgagcctagatgggctgttacacaggctggcgatcctggcgccgatgatgaaggacctctgctggaacctgtgggctctgaacatgcccaggacacctatctggtgctggacaagtggctgctccccagaacaccctgtagcgagaatctgtctggccctggcggatccgtggatcccgtgacaatggatgaggccagcgagacaagcagctgcccttctgatctggccagcaagcctagacctgagggcacaagccctagcagcttcgagtacaccattctggaccccagcagccagctgctgtgtcctagagcactgcctccagagctgcctcctacacctcctcacctgaagtacctgtacctggtggtgtccgacagcggcatcagcaccgattatagctctggtggctctcagggcgtgcacggcgatagttctgatggcccttactctcacccctacgaaaacagcctggtgcctgacagcgaacctctgcaccctggatacgtggcctgtagctaa

SEQ ID NO: 12 - protein, 498 aa, Artificial sequence
  Synthetic Sequence
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 13 - DNA, 1497 bp, Mus musculus
atggctaccatagctcttccctggagcctgtccctctacgtcttcctcctgctcctggctacaccttgggcatctgcagcagtgaaaaactgttcccatcttgaatgcttctacaactcaagagccaatgtctcttgcatgtggagccatgaagaggctctgaatgtcacaacctgccacgtccatgccaagtcgaacctgcgacactggaacaaaacctgtgagctaactcttgtgaggcaggcatcctgggcctgcaacctgatcctcgggtcgttcccagagtcccagtcactgacctccgtggacctccttgacataaatgtggtgtgctgggaagagaagggttggcgtagggtaaagacctgcgacttccatccctttgacaaccttcgcctggtggcccctcattccctccaagttctgcacattgatacccagagatgtaacataagctggaaggtctcccaggtctctgacttcattgaaccatacttggaatttgaggcccgtagacgtcttctgggccacagctgggaggatgcatccgtattaagcctcaagcagagacagcagtggctcttcttggagatgctgatccctagtacctcatatgaggtccaggtgagggtcaaagctcaacgaaacaataccgggacctggagtccctggagccagcccctgacctttcggacaaggccagcaagcgatctggaccctctgatcctgacactgagcctgatcctggtgctgatctccctgctgctgacagtgctggctctgctgagccacagaagaaccctgcagcagaagatctggcctggcatcccatctccagagagcgagttcgagggcctgttcaccacacacaagggcaacttccagctgtggctgctgcagcgagatggctgtctttggtggtcccctggctctagctttcctgaggaccctcctgctcacctggaagtgctgtctgagcctagatgggccgttacacaggctggcgatccaggcgctgatgatgaaggacctctgctggaacctgtgggctctgagcacgctcaggacacctatctggtgctggacaagtggctgctccccagaacaccttgctccgagaacctttctggccctggcggatctgtggaccctgtgacaatggacgaggccagcgagacaagcagctgtccttctgacctggccagcaagcctagacctgagggcacaagccctagcagcttcgagtacaccattctggaccccagcagccagctgctgtgtcctagagcactgcctccagagctgcctcctacacctcctcacctgaagtttctgtttctggtggtgtccgacagcggcatcagcaccgattatagctctggtggctctcagggcgtgcacggcgatagttctgatggcccttactctcacccctacgaaaacagcctggtgcctgacagcgagcctctgcaccctggatatgtggcctgtagctga

SEQ ID NO: 14 - protein, 498 aa, Artificial sequence
  Synthetic Sequence
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSDFIEPYLEFEARRRLLGHSWEDASVLSLKQRQQWLFLEMLIPSTSYEVQVRVKAQRNNTGTWSPWSQPLTFRTRPASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKFLFLVVSDSGISTDYSSGGSQGVHGDSSDGPYSHPYENSLVPDSEPLHPGYVACS

SEQ ID NO: 15 - DNA, 1656 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggtctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 16 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 17 - DNA, 1389 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 18 - protein, 462 aa, Artificial sequence
  Synthetic Sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPANNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 19 - DNA, 1644 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggtctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaaccaagaagaagcatatgtcaccatgtccagcttctaccaaaaccagtga

SEQ ID NO: 20 - protein, 547 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNQEEAYVTMSSFYQNQ

SEQ ID NO: 21 - DNA, 1496 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcacagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattcta

SEQ ID NO: 22 - protein, 498 aa, Artificial sequence
  Synthetic Sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 23 - DNA, 1638 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 24 - protein, 545 aa, Artificial sequence
  Synthetic Sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 25 - DNA, 1494 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 26 - protein, 497 aa, Artificial sequence
  Synthetic Sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPASDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 27 - DNA, 1494 bp, Homo sapiens
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagttcctgttccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 28 - protein, 497 aa, Artificial sequence
  Synthetic Sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPASDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKFLFLVVSDSGISTDYSSGDSQGAQGGLSDGPYSNPYENSLIPAAEPLPPSYVACS

SEQ ID NO: 29 - DNA, 510 bp, Mus musculus
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggacaacctgttggtgctgctaaaggccctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 30 - protein, 169 aa, Mus musculus
MYSMQLASCVTLTLVLLVNSAPTSSSTSSSTAEAQQQQQQQQQQQQHLDNLLVLLKALLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 31 - DNA, 2388 bp, Mus musculus
atgctactagtaaatcagtcacaccaaggcttcaataaggaacacacaagcaagatggtaagcgctattgttttatatgtgcttttggcggcggcggcgcattctgcctttgcgggatccaggggtgtgtttcgccgagaagcacacaagagtgagatcgcccatcggtataatgatttgggagaacaacatttcaaaggcctagtcctgattgccttttcccagtatctccagaaatgctcatacgatgagcatgccaaattagtgcaggaagtaacagactttgcaaagacgtgtgttgccgatgagtctgccgccaactgtgacaaatcccttcacactctttttggagataagttgtgtgccattccaaacctccgtgaaaactatggtgaactggctgactgctgtacaaaacaagagcccgaaagaaacgaatgtttcctgcaacacaaagatgacaaccccagcctgccaccatttgaaaggccagaggctgaggccatgtgcacctcctttaaggaaaacccaaccacctttatgggacactatttgcatgaagttgccagaagacatccttatttctatgccccagaacttctttactatgctgagcagtacaatgagattctgacccagtgttgtgcagaggctgacaaggaaagctgcctgaccccgaagcttgatggtgtgaaggagaaagcattggtctcatctgtccgtcagagaatgaagtgctccagtatgcagaagtttggagagagagcttttaaagcatgggcagtagctcgtctgagccagacattccccaatgctgactttgcagaaatcaccaaattggcaacagacctgaccaaagtcaacaaggagtgctgccatggtgacctgctggaatgcgcagatgacagggcggaacttgccaagtacatgtgtgaaaaccaggcgactatctccagcaaactgcagacttgctgcgataaaccactgttgaagaaagcccactgtcttagtgaggtggagcatgacaccatgcctgctgatctgcctgccattgctgctgattttgttgaggaccaggaagtgtgcaagaactatgctgaggccaaggatgtcttcctgggcacgttcttgtatgaatattcaagaagacaccctgattactctgtatccctgttgctgagacttgctaagaaatatgaagccactctggaaaagtgctgcgctgaagccaatcctcccgcatgctacggcacagtgcttgctgaatttcagcctcttgtagaagagcctaagaacttggtcaaaaccaactgtgatctttacgagaagcttggagaatatggattccaaaatgccattctagttcgctacacccagaaagcacctcaggtgtcaaccccaactctcgtggaggctgcaagaaacctaggaagagtgggcaccaagtgttgtacacttcctgaagatcagagactgccttgtgtggaagactatctgtctgcaatcctgaaccgtgtgtgtctgctgcatgagaagaccccagtgagtgagcatgttaccaagtgctgtagtggatccctggtggaaaggcggccatgcttctctgctctgacagttgatgaaacatatgtccccaaagagtttaaagctgagaccttcaccttccactctgatatctgcacacttccagagaaggagaagcagattaagaaacaaacggctcttgctgagctggtgaagcacaagcccaaggctacagcggagcaactgaagactgtcatggatgactttgcacagttcctggatacatgttgcaaggctgctgacaaggacacctgcttctcgactgagggtccaaaccttgtcactagatgcaaagacgccttagccggcggtggcggttcagcacccacttcaagctccacttcaagctctacagcggaagcacagcagcagcagcagcagcagcagcagcagcagcagcacctggataatctgttggtgctgctaaaggcgctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaagcggccgcgcatcatcaccaccatcaccaccattaa

SEQ ID NO: 32 - protein, 795 aa, Mus musculus
MLLVNQSHQGFNKEHTSKMVSAIVLYVLLAAAAHSAFAGSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSAPTSSSTSSSTAEAQQQQQQQQQQQQHLDNLLVLLKALLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQAAAHHHHHHHH

SEQ ID NO: 33 - DNA, 489 bp, Homo sapiens
atgtataggatgcagttgctcagttgtatagcactgtcccttgcgctggttacgaacagcgcacctacttcaagttctacaaagaaaacacagctacaactgagccaattacttgtgctgttaaaggcgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaactgcggccgcccaccatcaccatcaccattag

SEQ ID NO: 34 - protein, 155 aa, Homo sapiens
MYRMQLLSCIALSLALVTNSGSAPTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - DNA, 2346 bp, Homo sapiens
atgctactagtaaatcagtcacaccaaggcttcaataaggaacacacaagcaagatggtaagcgctattgttttatatgtgcttttggcggcggcggcgcattctgcctttgcgggatccaggggtgtgtttcgccgagaagcacacaagagtgagatcgcccatcggtataatgatttgggagaacaacatttcaaaggcctagtcctgattgccttttcccagtatctccagaaatgctcatacgatgagcatgccaaattagtgcaggaagtaacagactttgcaaagacgtgtgttgccgatgagtctgccgccaactgtgacaaatcccttcacactctttttggagataagttgtgtgccattccaaacctccgtgaaaactatggtgaactggctgactgctgtacaaaacaagagcccgaaagaaacgaatgtttcctgcaacacaaagatgacaaccccagcctgccaccatttgaaaggccagaggctgaggccatgtgcacctcctttaaggaaaacccaaccacctttatgggacactatttgcatgaagttgccagaagacatccttatttctatgccccagaacttctttactatgctgagcagtacaatgagattctgacccagtgttgtgcagaggctgacaaggaaagctgcctgaccccgaagcttgatggtgtgaaggagaaagcattggtctcatctgtccgtcagagaatgaagtgctccagtatgcagaagtttggagagagagcttttaaagcatgggcagtagctcgtctgagccagacattccccaatgctgactttgcagaaatcaccaaattggcaacagacctgaccaaagtcaacaaggagtgctgccatggtgacctgctggaatgcgcagatgacagggcggaacttgccaagtacatgtgtgaaaaccaggcgactatctccagcaaactgcagacttgctgcgataaaccactgttgaagaaagcccactgtcttagtgaggtggagcatgacaccatgcctgctgatctgcctgccattgctgctgattttgttgaggaccaggaagtgtgcaagaactatgctgaggccaaggatgtcttcctgggcacgttcttgtatgaatattcaagaagacaccctgattactctgtatccctgttgctgagacttgctaagaaatatgaagccactctggaaaagtgctgcgctgaagccaatcctcccgcatgctacggcacagtgcttgctgaatttcagcctcttgtagaagagcctaagaacttggtcaaaaccaactgtgatctttacgagaagcttggagaatatggattccaaaatgccattctagttcgctacacccagaaagcacctcaggtgtcaaccccaactctcgtggaggctgcaagaaacctaggaagagtgggcaccaagtgttgtacacttcctgaagatcagagactgccttgtgtggaagactatctgtctgcaatcctgaaccgtgtgtgtctgctgcatgagaagaccccagtgagtgagcatgttaccaagtgctgtagtggatccctggtggaaaggcggccatgcttctctgctctgacagttgatgaaacatatgtccccaaagagtttaaagctgagaccttcaccttccactctgatatctgcacacttccagagaaggagaagcagattaagaaacaaacggctcttgctgagctggtgaagcacaagcccaaggctacagcggagcaactgaagactgtcatggatgactttgcacagttcctggatacatgttgcaaggctgctgacaaggacacctgcttctcgactgagggtccaaaccttgtcactagatgcaaagacgccttagccggcggtggcggttcacccggggcacctacttcaagttctacaaagaaaacacagctacaactgagccaattacttgtgctgttaaaggcgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaactgcggccgcgcatcatcaccaccatcaccaccattaa

SEQ ID NO: 36 - protein, 781 aa, Artificial sequence
  Synthetic Sequence
MLLVNQSHQGFNKEHTSKMVSAIVLYVLLAAAAHSAFAGSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSPGAPTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTAAAHHHHHHHH

(19) United States
(12) Patent Application Publication
(10) Pub. No.: US 2022/0296644 A1
(43) Pub. Date: Sep. 22, 2022

(54) CHIMERIC ORTHOGONAL RECEPTOR PROTEINS AND METHODS OF USE

(71) Applicant: The Board of Trustees of the Leland Stanford Junior University, Stanford, CA (US)

(72) Inventors: Kenan Christopher Garcia, Menlo Park, CA (US); Leon Lih-Ren Su, Stanford, CA (US)

(21) Appl. No.: 17/641,688

(22) PCT Filed: Sep. 10, 2020
(86) PCT No.: PCT/US2020/050232

§ 371 Date: Mar. 9, 2022

Related U.S. Application Data
(60) Provisional application No. 62/898,917, filed on Sep. 11, 2019.

(51) Int. Cl. A61K 35/17; A61P 31/00; A61P 35/00; A61P 37/02; C07K 14/725; C07K 14/705; C07K 14/715

(52) U.S. Cl. CPC A61K 35/17; A61P 31/00; A61P 35/00; A61P 37/02; C07K 14/7051; C07K 14/70596; C07K 14/7155; C07K 2319/03

(57) ABSTRACT

Engineered orthogonal chimeric receptor/ligand pairs, and methods of use thereof, are provided.

### CROSS REFERENCE

This application claims priority to U.S. Provisional Application No. 62/898,917, filed Sep. 11, 2019 which is incorporated herein in its entirety for all purposes.

### BACKGROUND

The ability to manipulate receptors to bind and respond to modified ligands in a manner independent, or orthogonal, from the influence of the native ligands, is a significant challenge in protein engineering. Synthetic ligand-ortholog receptor pairs have been created that are orthogonal to the analogous natural interaction. Manipulation of the intracellular signaling pathways activated by orthogonal ligands is of great interest and is addressed herein.

Orthogonal ligands and receptors are disclosed in US Patent publication 2018/0228842A, and in the international patent application US2019/021451; each herein specifically incorporated by reference.

### SUMMARY

Engineered chimeric orthogonal receptors, and methods of use thereof, are provided herein. In the chimeric orthogonal receptors, an orthogonal ligand binding domain (oLBD) derived from a first receptor is operably linked to an intracellular domain (ICD) derived from a second receptor.

The oLBD comprises a modified extracellular domain (ECD) of a receptor such as, e.g., the extracellular domain of the CD122 IL-2 receptor. The ECD is modified to comprise sequence modifications that alter its binding specificity, such that the modified ECD binds to an orthogonal ligand, a modified counterpart of the native ligand for the receptor. Binding of the orthogonal counterpart ligand to the oLBD activates signaling via the ICD of the receptor and provides specificity for extracellular interactions with ligand The ICD transmits the activation signal to cytoplasmic components of signaling pathways, and provides signaling specificity for these intracellular interactions, e.g., through activation of specific signal transduction pathways such as, e.g., JAK, STAT, etc. This modular approach allows an orthogonal cytokine and oLBD pair to be used in combination with a variety of different ICDs, activating signaling pathways in accordance with the ICD and providing flexibility in engineering cells for a desired response.

An orthogonal ligand specifically binds to its counterpart oLBD. The oLBD exhibits significantly reduced binding to its endogenous ligand, including to the native counterpart of the orthogonal ligand. The orthogonal ligand exhibits significantly reduced binding to its endogenous receptors, including to the native counterpart of the orthogonal receptor. In some embodiments, the affinity of the orthogonal ligand for the orthogonal receptor is comparable to the affinity of the native ligand for the native receptor.

In some embodiments, engineered chimeric orthogonal receptors comprise an oLBD derived from a first receptor operably linked to the ICD of a second receptor through a transmembrane domain. In some embodiments the oLBD is fused to the transmembrane domain derived from the second receptor. In other embodiments the transmembrane domain is provided by the receptor from which the oLBD is derived. In other embodiments, the transmembrane may be an artificial amino acid sequence derived. In other embodiments, the transmembrane domain is derived from a third transmembrane protein.

In some embodiments the ICD of the chimeric orthogonal receptor is a functional fragment derived from a receptor, for example derived from a cytokine receptor. In some such embodiments the ICD is a functional fragment derived from a receptor and is substantially or entirely the ICD of the native receptor. In some embodiments the ICD comprises one or more amino acid substitutions relative to the ICD of the native receptor. In some embodiments the ICD of the chimeric receptor comprises binding sites for one or more STAT signaling proteins, e.g. STAT1, STAT2, STAT3, STAT4, STAT5A, STAT5B, STAT6, etc. In some embodiments the ICD of the chimeric receptor comprises one or more amino acid residues, e.g. a tyrosine residue, that is phosphorylated by an intracellular kinase (e.g. a JAK kinase).

The intracellular signaling pathway(s) activated in response to the binding of the orthogonal ligand to the oLBD of the chimeric orthogonal receptor results in an intracellular signaling pattern characteristic of the signaling pathway(s) invoked by activation of the parent receptor from which the ICD of the chimeric receptor is derived in response to the binding of native ligand to such parent receptor. For example, the specificity and/or pattern for activation of one or more STAT signaling proteins in response to the binding of the orthogonal ligand to the chimeric orthogonal receptor may be substantially the same as for activation of the native receptor from which the ICD is derived.

In some embodiments the orthogonal ligand is derived from a cytokine protein and the orthogonal receptor is derived from a cytokine receptor. In some embodiments the orthogonal cytokine is an orthogonal IL-2 protein. In some embodiments the orthogonal ligand is derived from the human IL-2 protein. In some such embodiments the orthogonal receptor is an orthogonal IL-2 receptor beta protein, also referred to as an orthogonal CD122 protein. In some embodiments, the extracellular domain of the chimeric orthogonal receptor is derived from human CD122. In some embodiments, the ECD of the chimeric orthogonal receptor comprises a polypeptide of SEQ ID NO: (insert number for orthoCD122). In some embodiments the ICD of the chimeric receptor comprises a polypeptide sequence derived from an ICD of a common gamma chain receptor (CD132) family member other than CD122. In some embodiments the ICD of the chimeric orthogonal receptor comprises the ICD of a common gamma chain receptor family member selected from group consisting essentially of the IL-4 receptor (IL4R, IL-4Rα, CD124), the IL-7 receptor (IL7R, IL-7Rα, CD127), the IL-9 receptor (IL-9R, CD129), the IL-15Rα (CD215), and the IL-21 receptor (IL-21R, CD360). In some embodiments, the ICD of the chimeric orthogonal receptor is derived from the ICD of the erythropoietin receptor (EpoR).

In some embodiments, an engineered cell is provided, in which the engineered cell has been modified by introduction of a chimeric orthogonal receptor of the invention, the chimeric orthogonal receptor comprising an oLBD from a first receptor operably linked through a transmembrane domain to an ICD derived from a second receptor. Any cell can be used for this purpose. In some embodiments the cell is a mammalian cell. In some embodiments the cell is a human cell. In some embodiments, the cell is a mammalian immune cell. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, αβ T cells, γδ T cells and engineered variants of such T cells including CAR T cells; tumor infiltrating lymphocytes (TILs), etc. In other embodiments the engineered cell is a stem cell, including but not limited to a hematopoietic stem cell, an NK cell, a macrophage, or a dendritic cell. In some embodiments the cell is genetically modified in an ex vivo procedure, prior to transfer into a subject, to introduce a coding sequence for the chimeric receptor. genetically engineered to express a chimeric orthogonal receptor and an engineered T cell receptor. Examples of engineered T cell receptors include but are not limited to chimeric antigen receptors, engineered TCR and the like. In some embodiments, the present invention provides a method of preparing a cell comprises a cell comprising a chimeric orthogonal receptor and an engineered T cell receptor, the method comprising the isolation of a cell from a subject and introducing into the isolated cell a nucleic acid sequence encoding an engineered T cell receptor, chimeric antigen receptor, etc. In some embodiments, the disclosure provides an engineered cell expressing a chimeric orthogonal receptor a cell (or population of cells) is obtained from a subject and genetically modified ex vivo to introduce a vector, the vector comprising a nucleic acid encoding a chimeric orthogonal receptor of the present disclosure and a engineered T cell receptor including but not limited to a chimeric antigen receptor (CAR). The engineered cell expressing the chimeric orthogonal receptor can be provided in a unit dose for therapy, and can be allogeneic, autologous, etc. with respect to an intended recipient.

In some embodiments, a vector comprising a polynucleotide coding sequence that encodes a chimeric orthogonal receptor of the invention is provided, where the coding sequence is operably linked to a promoter active in the desired cell for expression of the chimeric orthogonal receptor, where an active promoter may be constitutively active or may be regulated. Various vectors are known in the art and can be used for this purpose, e.g. replication competent, replication deficient or conditionally replicating viral vectors, plasmid vectors, minicircle vectors. In some embodiments, the vector maybe integrated into the target cell genome or can be episomally maintained.

The vectors provided herein may be provided in a kit, optionally combined with a orthogonal ligand or vector encoding an orthogonal ligand that binds to and activates the chimeric orthogonal receptor. In some embodiments the vector containing the coding sequence for the orthogonal ligand is operably linked to a high expression promoter active in a target cell. In other embodiments, a kit is provided in which the vector encoding the orthogonal chimeric receptor is provided with a purified composition of the orthogonal ligand, e.g. in a unit dose, packaged for administration to a patient (e.g. a prefilled syringe). In still some other embodiments, a kit is provided in which the vector encoding the chimeric orthogonal receptor is provided with a vector encoding the orthogonal ligand to enable expression of the chimeric orthogonal receptor in a cell and also expression of the orthogonal ligand intended for secretion by the same cell (or other cell) to enable autocrine, endocrine, or paracrine ligand/receptor signaling.

In some embodiments a therapeutic method is provided, the method comprising introducing into a subject in need thereof a therapeutically effective quantity of an engineered cell population, wherein all or a part of the cell population has been modified by introduction of a nucleic acid sequence encoding a chimeric orthogonal receptor of the invention. The cell population may be engineered ex vivo, and may be autologous or allogeneic with respect to the subject. In some embodiments, the introduced cell population is contacted with the cognate orthogonal ligand in vivo following administration of the engineered cells. In some embodiments the engineered cell is a T cell. In some embodiments the engineered cell is a CAR T cell.

### BRIEF DESCRIPTION OF THE DRAWINGS

The invention may be understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1, panel A provides a schematic of the crystal structure of IL2-IL2R complex and schematic diagrams of murine orthogonal IL-2Rβ (mIL2Rb) chimeric proteins illustrating one embodiment of the present disclosure, in particular illustrating (a) a murine ortho IL-2Rβ and IL2Rβ transmembrane and intracellular domains (“moRb (full length)”, SEQ ID NO:2), (b) a chimeric orthogonal recepto9r comprising the extracellular domain of a murine orthogonal IL-2Rβ (moRB ECD) and the transmembrane (TM) and intracellular domains of murine IL-7 receptor mil7ICD (SEQ ID NO:4), and (c) a chimeric orthogonal receptor comprising the extracellular, transmembrane and a sequence partial intracellular domain of the murine ortho IL-2Rβ and “mIL-7RpYtail”) (SEQ ID NO:6). Additionally, partial protein sequences of the chimeric receptors are provided illustrating that the C-termini with STAT5 signaling protein binding site (boxed), which binding site includes a tyrosine target residue (pY) for phosphorylation. FIG. 1, Panel B provides data from an experiment where T cells isolated from BL6 mice were activated by contacting with anti-CD3/anti-CD28 coated beads and transduced with recombinant retroviral vectors encoding the indicated chimeric or wild-type receptors, the retroviral construct containing an IRES sequence and yellow fluorescent protein (YFP). Transduced cells were stimulated with mouse ortho-IL2 (SEQ ID NO:30) for 15 minutes, then fixed in paraformaldehyde (PFA), methanol (MeOH) permeabilized and stained with anti-pSTAT5-A647 antibody. Samples were analyzed on a CytoFLEX® flow cytometer (Beckman Coulter Life Sciences, Indianapolis Ind.) gating on YFP+ cell data plotted with Prism® software (GraphPad Software, San Diego Calif. USA). SEM, n=3. The data provided demonstrate a change in the phosphorylation of STAT5, which varies according to the intracellular domain of the receptor.

FIG. 2 provides graphical representations of data generated from experiments to evaluate STAT5, STAT3 and STAT1 signaling in T cell blasts recombinantly modified to express areceptor comprising the murine ortho IL2 extracellular domains and the transmembrane and intracellular signaling domains of: the IL2 receptor beta subunit (moRb-IL2Rb, SEQ ID NO:2), the IL7 receptor transmembrane and intracellular domains (moRb-IL7, SEQ ID NO:4), the IL21 receptor transmembrane and intracullar domains (moRb-IL21, SEQ ID NO:10) and the IL9 receptor transmembrane and intracullar domains (moRb-IL9, SEQ ID NO:8) in response to exposure to the murine ortho-IL2 ligand (SEQ ID 30). T cells from BL6 mice were isolated, anti-CD3/anti-CD28 activated and transduced with the indicated moRb IRES YFP retrovirus (RV): moRb (SEQ ID NO:2), moRb-IL-7R (SEQ ID NO:4), moRb-IL21R (SEQ ID NO:10), mRb-IL-9R (SEQ ID NO:8). Transduced cells were stimulated with ortho IL2 (SEQ ID NO:30) for 20′, then fixed in PFA, MeOH permeabilized and stained with anti-pSTAT5-A647 antibody, anti-pSTAT3-A647 antibody, or anti-pSTAT1-A647 antibody. The samples were analyzed on a CytoFLEX® flow cytometer, gating on YFP+ cells and the data plotted with the assistance of the Prism® software. The data show that the fusion receptors provide phosphorylation of STAT1, 3 and 5 intracellular signaling characteristic of the phosphorylation pattern characteristic of the receptor from which the intracellular domain was derived while maintaining the same IL-2 orthogonal extracellular receptor domain.

FIG. 3 provides data resulting from ortho IL2 (SEQ ID NO:30) stimulation of T cell blasts transduced with a vector encoding chimeric receptor comprising the extracellular domain of murine ortho IL-2 and the transmembrane and intracellular signaling domains of the erythropoietin (EPO) receptor (moRb-EpoR) demonstrating that the fusion receptor is capable of intracellular signaling and activating pSTAT5, a signal characteristic of an activated EPO receptor. Briefly, T cells from BL6 mice were isolated, anti-CD3/anti-CD28 activated and transduced with indicated with retroviral expression vectors comprising a IRES bi-cistronic expression cassette, the first cistron comprising a nucleic acid sequence encoding the moRb-EpoR fusion receptor (SEQ ID NO:12) or moRb-EpoR-YF fusion receptor (SEQ ID NO:14) with, in each case, the second cistron comprising a nucleic acid sequence encoding YFP. Transduced cells were stimulated with a murine ortho IL2 for 20 minutes, then fixed in PFA, MeOH permeabilized and stained with anti-pSTAT5-A647. The samples were analyzed on a CytoFLEX® flow cytometer, gating on YFP+ cells and the data plotted with the assistance of the Prism® software. The data provided in FIG. 3 illustrates that STAT5 phosphorylation, a signal characteristic of the EPO receptor increases after ortho-IL2 stimulation of the ECD of the fusion receptor.

FIG. 4 is a graphical representation of data generated from experiments to demonstrate that ortho-IL-2 induces proliferation in T cells transduced with a recombinant retroviral encoding chimeric receptors. Briefly, T cells from BL6 mice were isolated, anti-CD3/anti-CD28 activated and transduced with indicated retrovirus: moRb (SEQ ID NO:2), moRb-EpoR (SEQ ID NO:12) or moRb-EpoR(YF) (SEQ ID NO:14). Cells were labeled with CellTrace™ Violet (CTV, Thermo Fisher Scientific) on day 0, and incubated with indicated concentration of ortho-IL2 (SEQ ID NO:30). On day 3, samples were analyzed on a CytoFLEX® flow cytomer, gating on live, YFP+ cells. The figure provides representative data from 4 replicates of the experiment. The data demonstrate an ortho-IL2 dose dependent increase proliferation of T cells.

FIG. 5 provides the results of experiments resulting from human PBMCs transduced a nucleic acid sequence encoding receptors comprising the ECD of a human ortho-IL2Rb (hoRb) receptors contacted with a orthogonal hIL2 ligand demonstrating that the orthogonal chimeric receptors confer distinct STAT activation characteristic of the receptor from which the ICD is derived in response to activation of the hoRb ECD with the hoIL2 ligand As more fully described herein human ortho-IL2Rb-ICD chimeric receptors were cloned into a pMSCV-IRES-YFP retrovirus (RV) plasmid. RV supernatants were produced in HEK293T cells by standard protocol, and used to transduce anti-CD3/28 activated human peripheral blood mononuclear cells (PBMCs). Panel A provides a graphical representation of mean fluorescence intensity (MFI, y-axis) representative of the induction of phospho-STAT5 (upper panel), phospho-STAT3 (middle panel) and phospho-STAT1 (lower panel in PBMCs expressing the orthogonal receptors comprising the orthogonal IL2Rb sequence ECD (hoRb) operably linked to the intracellular domain of CD122 (hoRb/2Rb) and two chimeric orthogonal receptors comprising the orthogonal IL2Rb sequence ECD (hoRb) operably linked to the intracellular domains of hIL7R (hoRb/7R) and hIL9R(hoRb/9R) in response to stimulation with varying concentrations (X-axis) of a human orthogonal IL2 ligand that binds to the hoRb ECD (hIL2 SQVLKA) for 20 minutes, then PFA fixed, MeOH permeabilized, and subjected to pSTAT phospho-staining and FACS analysis. Panel B provides a summary of the table of the relative STAT activation. As illustrated by this data, the binding of the orthogonal ligand to the ECD of the chimeric receptors results in a an intracellular signal characteristic of the receptor from which the intracellular domain was derived.

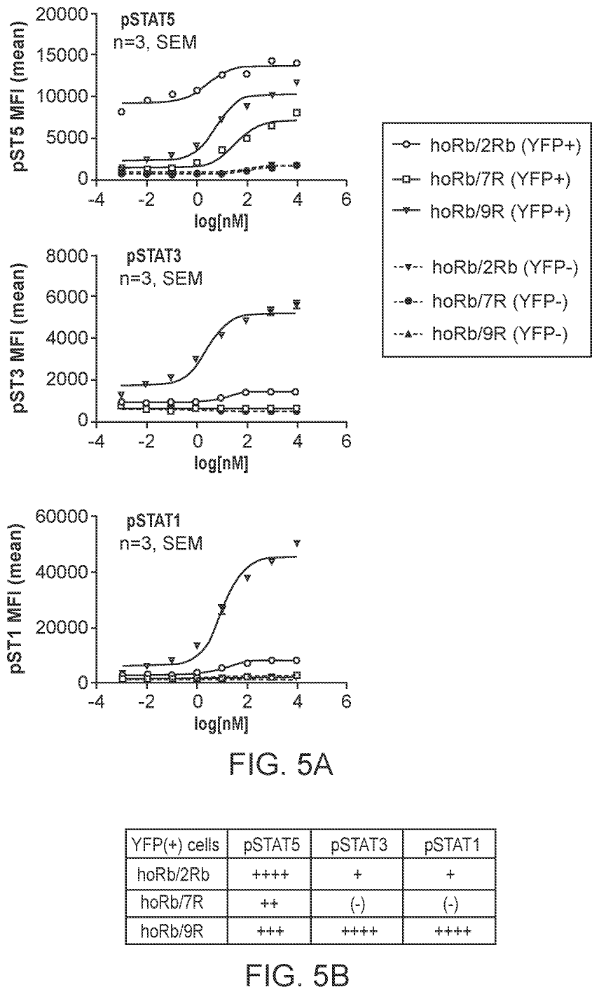

### DETAILED DESCRIPTION OF THE EMBODIMENTS

In order for the present disclosure to be more readily understood, certain terms and phrases are defined below as well as throughout the specification. The definitions provided herein are non-limiting and should be read in view of what one of skill in the art would know at the time of invention.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “polypeptide,” “protein” or “peptide” refer to any chain of amino acid residues, regardless of its length or post-translational modification (e.g., glycosylation or phosphorylation).

The term “identity,” as used herein in reference to polypeptide or DNA sequences, refers to the relative sequence identity between two molecules. The similarity between two amino acid or two nucleotide sequences is a direct function of the number of identical positions and is frequently expressed as a percentage (“percent identity”). In general, when determining identity of two sequences, the sequences are aligned so that the highest order match is obtained (greatest percent identity). Identity can be evaluated using published techniques and may be assessed using widely available computer programs, such as the GCS program package (Devereux et al., Nucleic Acids Res. 12:387, 1984), BLASTP, BLASTN, FASTA (Atschul et al., J. Molecular Biol. 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, the terms “protein variant” or “variant protein” or “variant polypeptide” and the like refer to a protein that differs from a reference polypeptide by virtue of at least one amino acid modification. The reference polypeptide may be a naturally occurring or wild-type (WT) polypeptide or may be a modified version of a WT polypeptide. In some embodiments, the variant polypeptide comprises at least one amino acid modification relative to a reference parent polypeptide. In some embodiments, the variant polypeptide comprises from about one to about ten amino acid modifications relative to a reference parent polypeptide. In some embodiments, the variant polypeptide comprises from about one to about five amino acid modifications compa relative to a reference parent polypeptide. In some embodiments, the variant polypeptide is at least about 99% identical to the reference protein, alternatively at least about 98% identical, alternatively at least about 97% identical, alternatively at least about 95% identical, alternatively at least about 90% identical. A variant protein may, for example, be at least about 99% identical to the reference protein, at least about 98% identical, at least about 97% identical, at least about 95% identical, at least about 90% identical to any one or more of SEQ ID NO:2; SEQ ID NO:4; SEQ ID NO:6; SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28.

As used herein, the terms “wild type” or “WT” or “native” refer to an amino acid sequence or a nucleotide sequence that is found in nature, including allelic variations. A wild-type polypeptide (e.g. protein, antibody, receptor, immunoglobulin, IgG, etc.) has an amino acid sequence or a nucleotide sequence that has not been modified by intervention of the hand of man.

The terms “recipient”, “individual”, “subject”, “host”, and “patient”, are used interchangeably herein and refer to any mammalian subject suffering from a disease, disorder or condition for whom diagnosis, treatment, or therapy is desired. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments the mammal is human.

As used herein, the term a “therapeutically effective amount” refers to that amount of the therapeutic agent sufficient to prevent, treat or manage the symptoms of a condition, disease or disorder. A therapeutically effective amount may refer to the amount of therapeutic agent sufficient to delay or minimize the onset of disease, e.g., delay or minimize the spread of cancer, or the amount effect to decrease or increase signaling from a receptor of interest. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prevent”, “preventing” and “prevention” refer to the prevention of the recurrence or onset of one or more symptoms of a disorder in a subject as result of the administration of a prophylactic or therapeutic agent.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with (e.g. simultaneously, in separate preparations or in a co-formulation, or in separate preparations the first provided agent administered to the subject within about 5 minutes of the administration of a second agent in the multiagent protocol), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Polypeptides

Ligands and receptors. The terms “orthogonal ligand”, “orthogonal receptor”, or “orthogonal ligand/receptor pair” refer to one or a pair of genetically engineered proteins that are modified by amino acid changes (including substitutions) such that an orthogonal ligand preferentially binds to an orthogonal receptor relative to the native (unmodified) receptor, and an orthogonal receptor preferentially binds to an orthogonal ligand relative to its native (unmodified) receptor.

An orthogonal ligand/receptor pair has been engineered by amino acid sequence changes relative to the native protein to (a) exhibit significantly reduced affinity to the native ligand or cognate receptor; and (b) to specifically bind to the counterpart engineered (orthogonal) ligand or receptor. Upon binding of the orthogonal ligand, the orthogonal receptor activates signaling that is transduced through native cellular elements to provide for a biological activity that mimics that native response, but which is specific to an engineered cell expressing the orthogonal receptor. An orthogonal receptor exhibits reduced binding to its cognate native ligand, while an orthogonal ligand exhibits significantly reduced binding to its cognate native receptor(s). In some embodiments the orthogonal ligand is orthogonal IL-2. In other embodiments the orthogonal ligand is an orthogonal variant of IL-15 or IL-7.

The process for engineering an orthogonal cytokine receptor pair may comprise the steps of: (a) engineering amino acid changes into a native receptor to disrupt binding to the native cytokine; (b) engineering amino acid changes into the native cytokine at contact residues for receptor binding, (c) selecting for cytokine orthologs that bind to the ortholog receptor; (d) discarding ortholog cytokines that bind to the native receptor, or alternatively to steps (c) and (d); (e) selecting for receptor orthologs that bind the ortholog cytokine; (f) discarding ortholog receptors that bind to the native cytokine. In preferred embodiments, knowledge of the structure of the cytokine/receptor complex is used to select amino acid positions for site-directed or error prone mutagenesis. Conveniently a yeast display system can be used for the selection process, although other display and selection methods are also useful.

As used herein, “significantly reduced binding” refers to little or no detectable binding and/or activation, or an insignificant level of binding and/or activation, e.g., to describe the comparative binding and activity of the orthogonal ligand relative to the naturally occurring ligand with respect to the naturally occurring receptor. The binding affinity may be, for example, determined with competitive binding experiments that measure the binding of a receptor with a single concentration of first labeled ligand in the presence of various concentrations of second unlabeled ligand. An orthogonal ligand exhibits significantly reduced binding with respect to the native form of the ligand if the orthogonal ligand binds to the native form of the receptor with less than 20%, alternatively less than about 10%, alternatively less than about 8%, alternatively less than about 6%, alternatively less than about 4%, alternatively less than about 2%, alternatively less than about 1%, alternatively less than about 0.5% of the level of binding of the naturally occurring ligand. Similarly, an orthogonal receptor exhibits significantly reduced binding with respect to the native form of the ligand if the native form of the ligand binds to the orthogonal form of the receptor with less than 20%, alternatively less than about 10%, alternatively less than about 8%, alternatively less than about 6%, alternatively less than about 4%, alternatively less than about 2%, alternatively less than about 1%, alternatively less than about 0.5% of the naturally occurring receptor.

An orthogonal ligand specifically binds to one or more cognate orthogonal receptors. The term “specifically binds” refers to the degree of selectivity or affinity for which one molecule binds to another. A first molecule of a binding pair may be said to specifically bind to a second molecule of a binding pair when the first molecule of the binding pair has a binding affinity for the second molecule at least 2 times greater, at least 10 times greater, at least 20 times greater, or at least 100 times greater than the affinity of the first molecule for other components present in a sample. Specific binding or affinity measurements may be assessed using techniques known in the art including but not limited to competition ELISA, BIACORE® assays and/or KINEXA® assays. The affinity of the orthogonal ligand for the cognate orthogonal receptor may be comparable to the affinity of the native ligand for the native receptor, in some embodiments having an affinity that is least about 5% of the native ligand receptor pair affinity, at least about 10%, at least about 15%, at least about 25%, at least about 50%, at least about 75%, at least about 100%, and may be higher, e.g. 2×, 3×, 4×, 5×, 10× or more of the affinity of the native ligand for the native receptor. Preferential binding may be, for example where the preference ratio is 5:1, 10:1, 20:1, etc.

Chimeric orthogonal receptors comprise an orthogonal ligand binding domain (oLBD) operably linked to an intracellular domain (ICD) derived from a receptor other than the receptor that from which the oLBD is derived. In some embodiments, the oLBD sequence is fused to the transmembrane domain of the protein from which the ICD is derived. In other embodiments the transmembrane domain is provided by the receptor from which the oLBD is derived; by an artificial sequence, derived from a third protein, etc. In some embodiments the ICD of the chimeric receptor is substantially or entirely the ICD of a native receptor. In some embodiments the ICD of the chimeric receptor comprises one or more amino acid substitutions relative to the ICD of the native receptor. In some embodiments the ICD of the chimeric receptor comprises binding sites for one or more STAT signaling proteins, e.g. STAT1, STAT2, STAT3, STAT4, STAT5A, STAT5B, STAT6, etc. In some embodiments the ICD of the chimeric receptor comprises an amino acid residue, e.g. a tyrosine residue, that is phosphorylated by a JAK kinase.

The intracellular signaling pathways activated by binding the orthogonal ligand to the chimeric receptor can reflect the signal characteristic pattern of activation of the ICD of the receptor from which the intracellular domain of the chimeric receptor is derived. For example the pattern for activation of selected STAT proteins may be substantially similar to the pattern of activation that results from activation of the native receptor from which the ICD is derived with its native ligand.

Exemplary human cytokine receptors from which the ICD may be derived include, without limitation, CD121α; CD121β; IL-18Rα; IL-18Rβ; CD122 (in combination with a ligand binding domain other than CD122 ligand binding domain); CD124; CD213; CD127; IL-9R; CD21α1; CD213α2; IL-15Rα; CDw131; CDw125; CD131; CD126; CD130; IL-11 Rα; CD114; CD212; LIFR; OSMR; CDw210; IL-20Rα, IL-20Rβ; IL-14R; CD4; CDw217; CD118; CD119; CD40; LTβR; CD120α; CD120β; CD137 (4-1BB); BCMA, TACI; CD27; CD30; CD95 (Fas); GITR; LTβR; HVEM; OX40; BCMA, TACI; TRAILR1-4; Apo3; RANK, OPG; TGF-βR1; TGF-βR2; TGF-βR3; EpoR; TpoR; Flt-3; CD117; CD115; CD136; etc.

In some embodiments the ICD of the chimeric receptor is derived from an ICD of a receptor other than CD122 that is associated with the common gamma chain (CD132). In some embodiments the ICD is the ICD of a receptor selected from IL-4 receptor (CD124), IL-7 receptor (IL-7R), IL-9 receptor (CD129), IL-15Rα, IL-21 receptor (IL-21 R). In some embodiments the ICD present in the chimeric receptor is the ICD of the erythropoietin receptor (EpoR).

In some specific embodiments an oLBD is operably linked to the transmembrane domain (TMD) and ICD of IL-7R, which chimeric receptor is exemplified by SEQ ID NO:4 and SEQ ID NO:18. SEQ ID NO:6 and SEQ ID NO:20 provide examples where the TMD and partial ICD is provided by CD122. Reference sequences for human IL-7R may be accessed at Genbank NP_002176. Relative to the reference sequence, the transmembrane domain comprises amino acid residues 240-264, and the ICD from residues 265-459. A construct of the invention may comprise, for example the TMD and ICD of the IL-7R reference sequence, from about residue 223, about residue 225, 230, 235, 240 to about residue 459, and in some embodiments comprises the terminal amino acids and the target tyrosine for JAK phosphorylation at residue 455.

In some specific embodiments an oLBD is operably linked to the transmembrane domain (TMD) and ICD of IL-9R, which chimeric receptor is exemplified by SEQ ID NO:8 and SEQ ID NO:22. Reference sequences for human IL-9R may be accessed at Genbank NP_002177. Relative to the reference sequence, the transmembrane domain comprises amino acid residues 271-291, and the ICD from residues 292-521. A construct of the invention may comprise, for example the TMD and ICD of the reference sequence, from about residue 255, about residue 257, 260, 265, 270, 271 to about residue 521.

In some specific embodiments an oLBD is operably linked to the transmembrane domain (TMD) and ICD of IL-21R, exemplified by SEQ ID NO:10 and SEQ ID NO:24. Reference sequences for human IL-21 R may be accessed at Genbank NP_068570. Relative to the reference sequence, the transmembrane domain comprises amino acid residues 233-253, and the ICD from residues 254-538. A construct of the invention may comprise, for example the TMD and ICD of the reference sequence, from about residue 225, about 230, about 233 to about residue 538.

In some specific embodiments an oLBD is operably linked to the transmembrane domain (TMD) and ICD of the erythropoietin receptor (EpoR), exemplified by SEQ ID NO:12 and SEQ ID NO:26. Reference sequences for human IL-21R may be accessed at Genbank NP_000112. Relative to the reference sequence, the transmembrane domain comprises amino acid residues 251-273, and the ICD from residues 274-508. A construct of the invention may comprise, for example the TMD and ICD of the reference sequence, from about residue 240, about 245, about 250, about 251 to about residue 508. A number of tyrosine residues have been indicated as important for phosphorylation and binding to STAT proteins, including residues 454, 456, 468, 489 and 504, which may be included in the ICD sequence.

In some specific embodiments an oLBD is operably linked to the transmembrane domain (TMD) and ICD of IL-4Rα. Reference sequences for human IL-4Rα may be accessed at Genbank NP_000409. Relative to the reference sequence, the transmembrane domain comprises amino acid residues 233-256, and the ICD from residues 257-825. A construct of the invention may comprise, for example the TMD and ICD of the reference sequence, from about residue 240, about 245, about 250, about 255, about 257 to about residue 825.

As indicated above, the transmembrane domain (TMD) of the chimeric receptor may be the TMD sequence of the same receptor protein from which the ICD is derived. Alternatively the transmembrane domain may comprise a polypeptide sequence which is thermodynamically stable in a eukaryotic cell membrane, long enough to span the membrane and typically composed of non-polar amino acids. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains are typically comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g., Pace, et al. (1998) Biophysical Journal 75: 422-427. Amino acids that are particularly favored in alpha helical conformations include methionine, alanine, leucine, glutamate, and lysine.

In some embodiments, the receptor that contributes the oLBD to the chimeric receptor is a chain of the IL-2 receptor, including but not limited to a polypeptide selected from interleukin 2 receptor beta (IL-2Rβ; also referred to as CD122), and interleukin 2 receptor gamma (IL-2Rγ; also referred to as CD132; also referred to as the “common gamma chain”). In some specific embodiments, the orthogonal receptor comprises a CD122 oLBD.

In some embodiments, the oLBD is a sequence variant of CD122. An exemplary oLBD for the human protein is SEQ ID NO:16, starting at amino acid residue 1 and including the sequence through residue 224. The ligand binding domain may further comprise the amino acid sequence up to residue 240, which is the start of the transmembrane domain, or a fraction thereof. For example a ligand binding domain may comprise or consist of residues starting 1-224, 1-225, 1-226, 1-227, 1-228, 1-229, 1-230, 1-231, 1-232, 1-233, 1-234, 1-235, 1-236, 1-237, 1-238, 1-239, 1-240, etc. of SEQ ID NO:16. Alternatively an orthogonal variant may be derived from the native protein sequence, e.g. Genbank accession number NP_000869, comprising or consisting of the sequence from 1-224, 1-225, 1-226, 1-227, 1-228, 1-229, 1-230, 1-231, 1-232, 1-233, 1-234, 1-235, 1-236, 1-237, 1-238, 1-239, 1-240, etc. For example, positions of interest for substitution or deletion include, without limitation, in human CD122 (hCD122) R41, R42, Q70, K71, T73, T74, V75, S132, H133, Y134, F135, E136, Q214.

An exemplary oLBD for the mouse protein is SEQ ID NO:2, starting at amino acid residue 1 and including the sequence through the cytokine binding motifs that are present at residue 224. The ligand binding domain may further comprise the amino acid sequence up to residue 240, which is the start of the transmembrane domain, or a fraction thereof. For example a ligand binding domain may comprise or consist of residues starting 1-224, 1-225, 1-226, 1-227, 1-228, 1-229, 1-230, 1-231, 1-232, 1-233, 1-234, 1-235, 1-236, 1-237, 1-238, 1-239, 1-240, etc. of SEQ ID NO:2. Alternatively an orthogonal variant may be derived from the native protein sequence, e.g. Genbank accession number NP_032394, comprising or consisting of the sequence from 1-224, 1-225, 1-226, 1-227, 1-228, 1-229, 1-230, 1-231, 1-232, 1-233, 1-234, 1-235, 1-236, 1-237, 1-238, 1-239, 1-240, etc. Positions of interest for substitution or deletion include, without limitation, in mouse CD122 (mCD122) R42, F67, Q71, S72, T74, S75, V76, S133, H134, Y135, I136, E137, and R215.

In some embodiments, CD122 is substituted at one or a combination of positions selected from Q71, T74, H134, Y135 in the mouse protein; or Q70, T73, H133, Y134 in the human protein. In some embodiments, the chimeric receptor comprises the ECD of CD122 comprising amino acid substitutions at mCD122 H134 and Y135; or hCD122 H133 and Y134. In some embodiments the amino acid substitution is to an acidic amino acid, e.g. aspartic acid and/or glutamic acid. Specific amino acid substitutions include, without limitation, mCD122 substitutions Q71Y; T74D; T74Y; H134D, H134E; H134K; Y135F; Y135E; Y135R; and hCD122 changes Q70Y; T73D; T73Y; H133D, H133E; H133K; Y134F; Y134E; Y134R. In some embodiments, the chimeric orthogonal receptor comprises an oLBD derived from human CD122 comprising amino acid subsitutions at H133 and Y134. In some embodiments, the chimeric orthogonal receptor comprises an oLBD derived from human CD122 comprising amino acid subsitutions at H133D and Y134F. In embodiments where the oLBD is an orthogonal CD122 protein, the orthogonal cytokine may be an orthogonal IL-2 polypeptide that exhibits significantly reduced activation of the native IL-2Rβ.

Interleukin 2 (IL-2) is a pluripotent cytokine produced primarily by activated CD4⁺ T cells and plays a crucial role in producing a normal immune response. Human IL-2 is synthesized as a precursor polypeptide of 153 amino acids, from which the n-terminal 20 amino acid signal peptide is removed post-translationally to generate mature secreted IL-2. A naturally occurring mature human IL-2 (hIL-2) occurs as a 133 amino acid sequence , as described in Fujita, et. al., PNAS USA, 80, 7437-7441 (1983). The amino acid sequence of human IL-2 is found in Genbank under accession locator NP_000577.2.

IL-2 activity may be measured, for example, in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The reference specific activity of recombinant human IL-2 is approximately 2.1×10⁴ IU/μg, which is calibrated against recombinant human IL-2 WHO International Standard (NIBSC code: 86/500). An orthogonal human IL-2 may have less than 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIBSC code: 86/500) human IL-2 polypeptide in a comparable assay.

An exemplary sequence for an orthogonal human IL-2 protein ligand is provided as SEQ ID NO:34. An exemplary sequence for orthogonal mouse IL-2 ligand is provided as SEQ ID NO:30. Alternatively an orthogonal protein may be designed based on the native human protein (refseq NP_000577.2) or the native mouse protein (NP_032392). In some embodiments, where the orthogonal ligand is a variant of IL2, one or more of the following amino acid residues are substituted with an amino acid other than that of the native protein, or are deleted at that position: for mouse IL-2 (mIL-2) any one of H27, L28, E29, Q30, M33, D34, Q36, E37, R41, N103; for human IL-2 (hIL-2) any one of Q13, L14, E15, H16, L19, D20, Q22, M23, G27, R81, N88. In some such embodiments, the set of sites for amino acid substitutions are selected from one or more of (for mIL-2) E29, Q30, M33, D34, Q36, and E37; and for hIL-2, E15, H16, L19, D20, Q22, M23, R81.

In some embodiments, the orthogonal ligand is a murine IL2 variant comprising one or more amino acid substitutions selected from: [H27W], [L28M, L28W], [E29D, E29T, E29A], [Q3ON], [M33V, M33I, M33A], [D34L, D34M], [Q36S, Q36T, Q36E, Q36K, Q36E], [E37A, E37W, E37H, E37Y, E37F, E37A, E37Y], [R41K, R415], [N103E, N103Q]. In some embodiments, the orthogonal ligand is human IL2 variant comprising one or more amino acid substitutions selected from: [Q13W], [L14M, L14W], [E15D, E15T, E15A, E15S], [H16N, H16Q], [L19V, L19I, L19A], [D2OL, D20M], [Q22S, Q22T, Q22E, Q22K, Q22E], [M23A, M23W, M23H, M23Y, M23F, M23Q, M23Y], [G27K, G27S], [R81D, R81Y], [N88E, N88Q], [T51I]. In some embodiments, the orthogonal ligand is a murine IL2 variant comprising a set of amino acid substitutions selected from one of the following sets of substitutions: [Q30N, M33V, D34N, Q36T, E37H, R41K]; [E29D, Q30N, M33V, D34L, Q36T, E37H]; [E29D, Q30N, M33V, D34L, Q36T, E37A], and [E29D, Q30N, M33V, D34L, Q36K, E37A], or a conservative variant thereof. In some embodiments, the orthogonal ligand is human IL2 variant comprising a set of substitutions selected from one of the following sets of subsitutions: [H16N, L19V, 20N, Q22T, M23H, G27K]; [E15D, H16N, L19V, D2OL, Q22T, M23H]; [E15D, H16N, L19V, D2OL, Q22T, M23A], and [E15D, H16N, L19V, D2OL, Q22K, M23A]; or a conservative variant thereof.

In some embodiments the orthogonal ligand is a human IL2 variant comprising amino acid substitutions for selected from one or more of: [E15S, E15T, E15Q, E15H]; [H16Q]; [L19V, L19I]; [D2OT, D20S, D20M, D20L]; [Q22K, Q22N]; [M23L, M23S, M23V, M23T]. In some embodiments, where the the orthogonal ligand is a human IL2 variant a consensus set of mutations for the orthogonal hIL-2 is [E15S, H16O, L19V, D20T/S/M; Q22K; M23L/S]. In some embodiments a consensus set of mutations for an orthogonal hIL-2 is [E15S, H16Q, L19V, D20L, M23 Q/A] and optionally Q22K.

In some embodiments the orthogonal ligand is human IL2 variant comprising a set of substitutions selected from one of the following sets of subsitutions: [E15S; H16Q; L19V, D20T/S; Q22K, M23L/S]; [E15S; H16Q; L19I; D20S; Q22K; M23L]; [E15S; L19V; D20M; Q22K; M23S]; [E15T; H16Q; L19V; D20S; M23S]; [E15Q; L19V; D20M; Q22K; M23S]; [E15Q; H16Q; L19V; D20T; Q22K; M23V]; [E15H; H16Q; L19V; D20S; Q22K; M23L]; [E15H; H16Q; L19I; D20L; Q22K; M23T]; [L19V; D20M; Q22N; M23S]; [E15S, H16Q, L19V, D20L, M23Q, R81D, T51I], [E15S, H16Q, L19V, D20L,M23Q, R81Y], [E15S, H16Q, L19V, D20L, Q22K, M23A], and [E15S, H16Q, L19V, D20L, M23A]. In some embodiments the orthogonal ligand is human IL2 variant comprising the substitutions E15S, H16Q, L19V, D20L, Q22K, M23A.

In some embodiments, an orthogonal ligand protein can be conjugated to additional molecules to provide desired pharmacological properties, such as extended half-life. In one embodiment, an orthogonal ligand is fused to the Fc domain of IgG, albumin (including human serum albumin), or other molecules to extend its half-life, e.g. by pegylation, glycosylation, and the like as known in the art. In some embodiments the orthogonal ligand is conjugated to a polyethylene glycol molecules or “PEGylated.” The molecular weight of the PEG conjugated to the orthogonal ligand includes but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa, in some embodiments the PEG has a molecular weight of approximately 70 kDa, in some embodiments the PEG has a molecular weight of approximately 80 kDa. In some embodiments, the PEG has an average molecular mass from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the orthogonal polypeptide ligand or via a linker molecule. The processes and chemical reactions necessary to achieve PEGylation of biological compounds are well known in the art.

In addition to extending the serum half-life, Fc-fusion can also endow the fusion partner with alternative Fc receptor mediated properties in vivo. An “Fc region” can be a naturally occurring or synthetic polypeptide that is homologous to an IgG C-terminal domain produced by digestion of IgG with papain. The orthogonal ligands can be fused to the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides. For example, see Wang X, Mathieu M, Brerski R J. IgG Fc engineering to modulate antibody effector functions. Protein Cell. 2018; 9(1):63-73. doi:10.1007/s13238-017-0473-8.

In other embodiments, an orthogonal ligand can comprise a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the chimeric polypeptide further comprises a C-terminal c-myc epitope tag. Ligands can also be synthesized with a HIS-tag, as known in the art, for ease in purification.

In some embodiments, the orthogonal ligand, e.g. orthogonal IL-2, can be acetylated. In some embodiments, the acetylation may occur at the N-terminus, using methods known in the art, e.g. by enzymatic reaction with N-terminal acetyltransferase and, for example, acetyl CoA. In some embodiments, the orthogonal ligand may be acetylated at one or more lysine residues, e.g. by enzymatic reaction with a lysine acetyltransferase. See, for example Choudhary et al. (2009). Science. 325 (5942): 834-840.

Orthogonal cytokine ligands and orthogonal chimeric receptors may include conservative modifications and substitutions at other positions of the polypeptide (e.g. positions other than those involved in the orthogonal engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ALA, PRO, GLY, GLN, ASN, SER, THR; Group II: CYS, SER, TYR, THR; Group III: VAL, ILE, LEU, MET, ALA, PHE; Group IV: LYS, ARG, HIS; Group V: PHE, TYR, TRP, HIS; and Group VI: ASP, GLU. In each instance, the introduction of additional modifications may be evaluated to minimize any increase in antigenicity of the modified polypeptide in the organism to which the modified polypeptide is to be administered.

### Nucleic Acids and Expression

In the present methods, an orthogonal protein may be produced by recombinant methods. A nucleic acid sequence encoding the orthogonal chimeric receptor or ligand may be incorporated into an expression vector in operable association with one or more expression control sequences (e.g. promoters, enhancers) into the cell to be engineered. The nucleic acid sequence encoding an orthogonal ligand or chimeric orthogonal receptor may be obtained from various sources as designed during the engineering process. Exemplary nucleic coding sequences are provided as SEQ ID NO:1, 3, 5, 7, 9, 11, 13, 15, 17, 19, 21, 23, 25, 27, 29, 31, 33, and 35, which may be provided as a ssDNA, dsDNA, DNA:RNA hybrid, ssRNA, dsRNA, or analogs thereof.

The orthogonal chimeric receptor or ligand and variants thereof may be prepared by introducing appropriate nucleotide changes into the coding sequences, as described herein. Such variants comprise insertions, substitutions, and/or deletions of, residues as noted. Any combination of insertion, substitution, and/or specified deletion is made to arrive at the final construct, provided that the final construct possesses the desired biological activity as defined herein.

To achieve expression of the recombinant protein, a nucleic acid encoding an orthogonal protein is inserted into a replicable vector for expression. Many such vectors are available. The vector components generally include, but are not limited to, one or more of the following: an origin of replication, an internal ribosome entry site (IRES), one or more marker genes, an enhancer element, a promoter, and a transcription termination sequence. Vectors include viral vectors, plasmid vectors, integrating vectors, and the like.

Expression vectors for expression of the orthogonal receptor may be viral vectors or non-viral vectors. Plasmids are examples of non-viral vectors. In order to facilitate transfection of the target cells, the target cell may be exposed directly to the non-viral vector under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, electroporation, and magnetic fields (electroporation). For example, see Novickij et al. (2016) Scientific Reports volume 6, Article number: 33537, “Pulsed Electromagnetic Field Assisted in vitro Electroporation”. In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. When a viral vector system is to be employed, retroviral, e.g. lentiviral expression vectors, are preferred. In particular, the viral vector is a gamma retrovirus ((Pule, et al. (2008) Nature Medicine 14(11):1264-1270), self-inactivating lentiviral vectors (June, et al. (2009) Nat Rev Immunol 9(10):704-716) and retroviral vectors as described in Naldini, et al. (1996) Science 272: 263-267; Naldini, et al. (1996) Proc. Natl. Acad. Sci. USA Vol. 93, pp. 11382-11388; Dull, et al. (1998) J. Virology 72(11):8463-8471; Milone, et al. (2009) 17(8):1453-1464; Kingsman, et al. U.S. Pat. No. 6,096,538 issued Aug. 1, 2000 and Kingsman, et al. U.S. Pat. No. 6,924,123 issued Aug. 2, 2005. In one embodiment, the expression vector is a Lentivector® lentiviral vector available from Oxford Biomedica.

Viral vectors of interest also include retroviral vectors (e.g. derived from MoMLV, MSCV, SFFV, MPSV, SNV etc), adeno-associated virus (AAV) vectors, adenoviral vectors (e.g. derived from Ad5 virus), SV40-based vectors, Herpes Simplex Virus (HSV)-based vectors etc.

Transduction of cells with an expression vector may be accomplished using techniques well known in the art including but not limited co-incubation with host T cells with viral vectors, electroporation, and/or chemically enhanced delivery.

An orthogonal protein may also be produced as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors may contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to an orthogonal protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication. Examples of promoters useful in the practice of the present disclosure include the CMV, EF-1, hPGK and RPBSA promoters.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, alpha-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryote, yeast, or higher eukaryote cells described above. Examples of useful mammalian host cell lines are mouse L cells (L-M[TK-], ATCC#CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, can be transfected with the above-described expression vectors. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression and will be apparent to the ordinarily skilled artisan.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in the same protein-coding open reading frame. However, enhancers do not have to be contiguous or in frame.

### Engineered Cells

In some embodiments, an engineered cell is provided, in which the cell has been modified by introduction of a expression vector comprising a nucleic acid sequence encoding a chimeric receptor of the invention, the chimeric receptor comprising an orthogonal ligand binding domain from a first receptor operably linked through a transmembrane domain to an intracellular domain (ICD) from a second receptor. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NK T cells, γδ T cells and engineered variants of such T cells including CAR T cells; etc. In other embodiments the engineered cell is a stem cell, e.g. a hematopoietic stem cell, an NK cell, a macrophage, or a dendritic cell. In some embodiments the cell is genetically modified in an ex vivo procedure, prior to transfer into a subject, to introduce a coding sequence for the chimeric receptor. The engineered cell can be provided in a unit dose for therapy, and can be allogeneic, autologous, or xenogeneic with respect to an intended recipient.

T cells useful for engineering with the constructs described herein include naïve T cells, central memory T cells, effector memory T cells or combination thereof. T cells for engineering as described above can be collected from a subject or a donor, and may be separated from a mixture of cells by techniques that enrich for desired cells or may be engineered and cultured without separation. An appropriate solution may be used for dispersion or suspension of the cells. Such solution will generally be a sterile balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. Techniques for affinity separation may include magnetic separation, using antibody-coated magnetic beads, affinity chromatography, cytotoxic agents linked to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42(4):626-39 “Adoptive Cell Therapy-Tumor-Infiltrating Lymphocytes, T cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

In some embodiments, an engineered T cell is allogeneic with respect to the individual that is treated, e.g. see clinical trials NCT03121625; NCT03016377; NCT02476734; NCT02746952; NCT02808442. See for review Graham et al. (2018) Cells. 7(10) E155. In some embodiments an allogeneic engineered T cell is fully HLA matched.

Allogeneic T cells may be genetically modified to reduce graft versus host disease. For example the engineered cells may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore the TRAC locus has been deleted for this purpose. A number of different approaches have been used to accomplish this deletion, e.g. CRISPR/Cas9; meganuclease; engineered I-Crel homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9) without directly incorporating the CAR into the TRAC loci. An alternative strategy to prevent GVHD modifies T cells to express an inhibitor of TCRαβ signaling, for example using a truncated form of CD3ζ as a TCR inhibitory molecule.

The preparation of T cells useful in the practice of the present invention is achieved by transforming isolated T cells with an expression vector comprising a nucleic acid sequence encoding the orthogonal chimeric receptor; optionally in combination with a nucleic acid sequence encoding a CAR polypeptide described below. The nucleic acid sequences encoding a CAR and an orthogonal chimeric receptor may each be provided on separate expression vectors, each nucleic acid sequence being operably linked to one or more expression control elements to achieve expression of the CAR and orthogonal receptor in the target cell, the vectors being co-transfected into the target cell. Alternatively, the nucleic acid sequences encoding the CAR and the orthogonal receptor may each be provided on a single vector each nucleic acid sequence under the control of one or more expression control elements to achieve expression of the associated nucleic acid sequence. Alternatively, both nucleic acid sequences may be under the control of a single promoter with intervening (e.g. T2A or IRES element) or downstream control elements that facilitate co-expression of the two sequences from the vector.

Ex vivo T cell activation may be achieved by procedures well-established in the art including cell-based T cell activation, antibody-based activation or activation using a variety of bead-based activation reagents. Cell-based T cell activation may be achieved by exposure of the T cells to antigen presenting cells, such as dendritic cells or artificial antigen presenting cells such as irradiated K562 cells. Antibody based activation of T cell surface CD3 molecules with soluble anti-CD3 monoclonal antibodies and soluble anti-CD28 antibodies also supports T cell activation.

T cells may be expanded by culturing the cells in contact with a surface providing an agent that stimulates a CD3 TCR complex associated signal (e.g., an anti-CD3 antibody) and an agent that stimulates a co-stimulatory molecule on the surface of the T cells (e.g an agonistic anti-CD28 antibody). Bead-based T cell activation has gained acceptance in the art for the preparation of T cells for clinical use. Bead-based activation of T cells may be achieved using commercially available T cell activation reagents including but not limited to the Invitrogen® CTS Dynabeads® CD3/28 (Life Technologies, Inc. Carlsbad Calif.) or Miltenyi MACS® GMP ExpAct Treg beads or Miltenyi MACS GMP TransAct™ CD3/28 beads (Miltenyi Biotec, Inc.). Conditions appropriate for T cell culture are well known in the art. Lin, et al. (2009) Cytotherapy 11(7):912-922; Smith, et al. (2015) Clinical & Translational Immunology 4:e31 published online 16 Jan. 2015. The target cells are maintained under conditions necessary to support growth, for example, an appropriate temperature (e.g., 37° C.) and atmosphere (e.g., air plus 5% CO₂).

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷cells/kg, at least 1×10⁸ cells/kg, at least 1×10⁸ cells/kg, at least 1×10¹⁰ cells/kg, or more, usually being limited by the number of T cells that are obtained during collection.

In one embodiment, a T cell expressing the orthogonal chimeric receptor is a T cell which has been modified to surface express a chimeric antigen receptor (a ‘CAR T’ cell). As used herein, the terms “chimeric antigen receptor T cell” and “CAR T cell” are used interchangeably to refer to a T cell that has been recombinantly modified to express a chimeric antigen receptor. As used herein, the terms “chimeric antigen receptor” and “CAR” are used interchangeably to refer to a polypeptide comprising multiple functional domains arranged from amino to carboxy terminus in the sequence: (a) an antigen binding domain (ABD), (b) a transmembrane domain (TM); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. Examples of commercially available CAR T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

As used herein, the term antigen binding domain (ABD) refers to a polypeptide that specifically binds to an antigen expressed on the surface of a target cell. The ABD may be any polypeptide that specifically binds to one or more antigens expressed on the surface of a target cell. In certain embodiments, the target cell antigen is a tumor antigen. Non-limiting examples of tumor antigens that may be targeted by a CAR include one or more antigens selected from the group including, but not limited to, the CD19, CD20, CD30, HER2, IL-11 Ra, PSCA, NCAM, NY-ESO-1, MUC1, CD123, FLT3, B7-H3, CD33, IL1RAP, CLL1 (CLEC12A)PSA, CEA, VEGF, VEGF-R2, CD22, ROR1, mesothelin, c-Met, Glycolipid F77, FAP, EGFRvIII, MAGE A3, 514, WT1, KG2D ligand, a folate receptor (FRa), GD2, PSMA, BCMA, and Wnt1 antigens.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS(USA) 85:5879-5883; S-z Hu, et al. (1996) Cancer Research, 56, 3055-3061. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S. Antibodies used in the preparation of scFvs may be optimized to select for those molecules which possess particular desirable characteristics (e.g. enhanced affinity) through techniques well known in the art such as phage display and directed evolution. In some embodiments, the ABD comprises an anti-CD19 scFv (see e.g., Cooper, et al., U.S. Pat. No. 9,701,758 issued Jul. 11, 2017, in particular the scFv FMC63 described therein), an anti-PSA scFv, an anti-PSMA scFv (see, e.g. Han, et al (2016) Oncotarget 7(37):59471-59481), an anti-BCMA scFv (see, e.g. the scFv antigen binding domains described in Brogdon, et al. U.S. Pat. No. 10,174,095 issued Jan. 8, 2019), an anti-HER2 scFv, an anti-CEA scFv, an anti-EGFR scFv, an anti-EGFRvIII scFv, an anti-NY-ESO-1 scFv, an anti-MAGE scFv, an anti-5T4 scFv, or an anti-Wnt1 scFv. In another embodiment, the ABD is a single domain antibody (also referred to as VHH) derived from anbitodies obtained through immunization of a camelid (e.g. a camel or llama) with a target cell derived antigen, in particular a tumor antigen. See, e.g. Muyldermans, S. (2001) Reviews in Molecular Biotechnology 74: 277-302. Alternatively, the ABD may be generated wholly synthetically through the generation of peptide libraries and isolating compounds having the desired target cell antigen binding properties in substantial accordance with the teachings or Wigler, et al. U.S. Pat. No. 6303313 B1 issued Nov. 12, 1999; Knappik, et al., U.S. Pat. No. 6,696,248 B1 issued Feb. 24, 2004, Binz, et al. (2005) Nature Biotechnology 23:1257-1268, and Bradbury, et al. (2011) Nature Biotechnology 29:245-254.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann (2012) MAbs. 4(2): 182-197; Stamova et al. (2012) Antibodies, 1(2), 172-198; Farhadfar et al. (2016) Leuk Res. 49:13-21; Benjamin et al. Ther Adv Hematol (2016) 7(3):142-56; Kiefer et al. Immunol Rev. (2016) 270(1):178-92; Fan et al. (2015) J Hematol Oncol. 8:130; May et al. (2016) Am J Health Syst Pharm. 73(1):e6-e13. In some embodiments, the chimeric bispecific binding member is a bivalent single chain polypeptides. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015. In some instances, a chimeric bispecific binding member may be a bispecific T cell engager (BiTE). A BiTE is generally made by fusing a specific binding member (e.g., a scFv) that binds an antigen to a specific binding member (e.g., a scFv) with a second binding domain specific for a T cell molecule such as CD3. In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137(8):2832-5; Ma et al. (2016) Proc Natl Acad Sci U S A. 113(4):E450-8 and Cao et al. (2016) Angew Chem Int Ed Engl. 55(26):7520-4.

In some embodiments, a linker polypeptide molecule is optionally incorporated into the CAR between the antigen binding domain and the transmembrane domain to facilitate antigen binding. Moritz and Groner (1995) Gene Therapy 2(8) 539-546. In one embodiment, the linker is the hinge region from an immunoglobulin, e.g. the hinge from any one of IgG1, IgG2a, IgG2b, IgG3, IgG4, particularly the human protein sequences. Alternatives include the CH2CH3 region of immunoglobulin and portions of CD3. In those instances where the ABD is an scFv, an IgG hinge may be employed. In some embodiments the linker comprises the amino acid sequence (G₄S)ₙ where n is 1, 2, 3, 4, 5, etc., and in some embodiments n is 3.

CARs useful in the practice of the present invention further comprise a transmembrane (TM) domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having a to favor alpha-helical conformations are well known in the art. See, e.g. Pace, et. al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3ζ, CD4, CD8, CD28, etc.

The cytoplasmic domain of the CAR polypeptide comprises one or more intracellular signal domains. In one embodiment, the intracellular signal domains comprise the cytoplasmic sequences of the T cell receptor (TCR) and co-receptors that initiate signal transduction following antigen receptor engagement and functional derivatives and sub-fragments thereof. A cytoplasmic signaling domain, such as those derived from the T cell receptor -chain, is employed as part of the CAR in order to produce stimulatory signals for T lymphocyte proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of P13 kinase, the human CD3ζ- chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T cell transduction, such as CD2, CD5 and CD28.

In some embodiments, the CAR may also provide a co-stimulatory domain. The term “co-stimulatory domain”, refers to a signaling endodomain of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the antigen-specific B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεRIγ, or the CD3ζ chain. The single signaling domain contains one or three immunoreceptor tyrosine-based activating motif(s) [ITAM(s)] for antigen-dependent T cell activation. The ITAM-based activating signal endows T cells with the ability to lyse the target tumor cells and secret cytokines in response to antigen binding. Second-generation CARs include a co-stimulatory signal in addition to the CD3ζ domain. Coincidental delivery of the delivered co-stimulatory signal enhances persistence, cytokine secretion and antitumor activity induced by CAR Transduced T cells. The co-stimulatory domain is usually located membrane proximal relative to the CD3ζ domain. Third-generation CARs include a tripartite signaling domain, comprising for example a CD28, a CD3ζ, and a OX40 or 4-1BB signaling region. Fourth generation CARs, or “armored car” CAR T cells are further gene modified to express or block molecules and/or receptors to enhance immune activity.

Exemplary intracellular signaling domains that may be incorporated into the CAR disclosed herein comprise (amino to carboxy): CD3ζ; CD28-41BB-CD3ζ; CD28-CD3ζ; CD28-OX40-CD3ζ; CD28-41BB-CD3ζ; 41BB-CD-28--CD3ζ and 41BB-CD3ζ.

The term CAR includes CAR variants including but not limited split CARs, ON-switch CARS, bispecific or tandem CARs, inhibitory CARs (iCARs) and induced pluripotent stem (iPS) CAR T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013) 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The terms “bispecific or tandem CARs” refer to CARs which include a secondary CAR binding domain that can either amplify or inhibit the activity of a primary CAR. In one embodiment, the ABD may comprise multiple (2, 3, 4 or more) binding domains such as multiple scFvs, antibodies, VHHs and combinations thereof, each of which binding domain specifically binds to a surface expressed molecule on the target cell. In one embodiment, the extracellular ABD domain of the CAR comprises a tandem bi-functional construct comprising a scFv that binds to CD19 operably linked to an scFv that binds to CD20.

The terms “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. Inhibitory CARs (iCARs) are designed to regulate CAR T cell activity through inhibitory receptors signaling modules activation. This approach combines the activity of two CARs, one of which generates dominant negative signals limiting the responses of CAR T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes, leading to less cytokine production, less efficient target cell lysis, and altered lymphocyte motility.

The terms “tandem CAR” or “TanCAR” refer to CARs which mediate bispecific activation of T cells through the engagement of two chimeric receptors designed to deliver stimulatory or costimulatory signals in response to an independent engagement of two different tumor associated antigens.

### Polypeptide Formulations

Recombinantly produced orthogonal ligands, e.g., for use with an engineered cell comprising an orthogonal chimeric receptor, can be recovered from culture medium of cells as a secreted polypeptide, although it can also be recovered from host cell lysates. A protease inhibitor, such as phenyl methyl sulfonyl fluoride (PMSF) also may be useful to inhibit proteolytic degradation during purification, and antibiotics may be included to prevent the growth of adventitious contaminants. Various purification steps are known in the art and find use, e.g. affinity chromatography. Size selection steps may also be used, e.g. gel filtration chromatography (also known as size-exclusion chromatography or molecular sieve chromatography) is used to separate proteins according to their size.

The orthogonal cytokine composition may be concentrated, filtered, dialyzed, etc., using methods known in the art. For therapeutic applications, the orthogonal ligands can be administered to a mammal comprising cells engineered to express an appropriate engineered orthogonal chimeric receptor to which the orthogonal ligand exhibits specific binding. Administration of the orthogonal ligand may be intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The orthogonal ligands also are suitably administered by intratumoral, peritumoral, intralesional, or perilesional routes or to the lymph, to exert local as well as systemic therapeutic effects.

Such dosage forms encompass physiologically acceptable carriers that are non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The polypeptide will typically be formulated in such vehicles at a concentration of about 0.1 μg/ml to 100 μg/ml.

In the event the orthogonal ligands are “substantially pure,” they can be at least about 60% by weight (dry weight) the polypeptide of interest, for example, a polypeptide containing the ortholog IL-2 amino acid sequence. For example, the polypeptide can be at least about 75%, about 80%, about 85%, about 90%, about 95% or about 99%, by weight, the polypeptide of interest. Purity can be measured by any appropriate standard method, for example, column chromatography, polyacrylamide gel electrophoresis, or HPLC analysis.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition is the orthogonal cytokine. The label on, or associated with, the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

### Therapeutic Cell Formulations and Uses

Methods and compositions are provided for enhancing cellular responses, by engineering cells from a recipient or donor by introduction of an orthogonal chimeric receptor of the invention, and stimulating the orthogonal chimeric receptor by contacting the engineered cell with the cognate orthogonal ligand that specifically binds to the oLBD and activates the chimeric receptor resulting in an intracellular signal. As discussed above, the subject methods include a step of obtaining the targeted cells, e.g. T cells, hematopoietic stem cells, etc., which may be isolated from a biological sample, or may be derived in vitro from a source of progenitor cells. The cells are transduced or transfected with an expression vector comprising a sequence encoding the orthogonal receptor, which step may be performed in any suitable culture medium. In some embodiments, a population of cells is obtained from a subject and genetically modified ex vivo to introduce a nucleic acid (e.g. a vector) comprising a nucleic acid sequence encoding a chimeric receptor operably linked to one or more expression control sequences functional in the isolated cell and the genetically modified cell is reintroduced into the subject from which it was obtained. In some embodiments, the present disclosure provides a method of autologous TIL cell therapy, the method providing isolating a population of tumor infiltrating lymphocytes (TILs) from a subject suffering from a neoplastic disease, a fraction (e.g. greater than 10%, optionally greater than 20%, optionally greater than 30%, optionally greater than 40%, optionally greater than 50%, optionally greater than 60%, optionally greater than 70%, optionally greater than 80%, or optionally greater than 90%) of the isolated TILs are genetically modified ex vivo by introducing a nucleic acid (e.g. a vector) company cellrising a nucleic acid sequence encoding a chimeric orthogonal receptor into said isolated TILs, and the generically modified TILs are reintroduced into the subject from which the cells were obtained. In some embodiments, the present disclosure provides a method of autologous TIL cell therapy, the method providing isolating a population of tumor infiltrating lymphocytes (TILs) from a subject suffering from a neoplastic disease, activating the TILs a fraction (e.g. greater than 10%, optionally greater than 20%, optionally greater than 30%, optionally greater than 40%, optionally greater than 50%, optionally greater than 60%, optionally greater than 70%, optionally greater than 80%, or optionally greater than 90%) of the isolated TILs are genetically modified ex vivo by introducing a nucleic acid (e.g. a vector) comprising a nucleic acid sequence encoding a chimeric orthogonal receptor into said isolated TILs, and the generically modified TILs are reintroduced into the subject from which the cells were obtained. In some embodiments, a cell is obtained from a first subject and genetically modified ex vivo to introduce a nucleic acid comprising a coding sequence for a chimeric orthogonal receptor and the generically modified cell is reintroduced into a different subject from which it was obtained (allogeneic cell transplant). In some embodiments, the present disclosure provides a cell

In some embodiments a therapeutic method is provided, the method comprising introducing into a recipient in need thereof of an engineered cell population, wherein the cell population has been modified by introduction of a vector comprising a sequence encoding an orthogonal chimeric receptor. The cell population may be engineered ex vivo, and is usually autologous or allogeneic with respect to the recipient. In some embodiments, the introduced cell population is contacted with the cognate orthogonal cytokine in vivo, following administration of the engineered cells.

Without being bound by theory, cells expressing an orthogonal chimeric receptor are selectively activated by an orthogonal ligand which has low affinity for and therefore results in low intracellular signaling activity from non-orthologous receptors. The specificity of the resulting activation of signaling pathways in the cell is determined by the TM and the ICD. In some embodiments the signaling pathways that are being activated are substantially similar to the signaling pathways activated by the receptor from which the ICD is derived, for example in the activity of specific JAK/STAT proteins. Extracellular binding of cytokines or growth factors induce activation of receptor-associated Janus kinases (JAKs), which phosphorylate a specific tyrosine residue within the STAT protein promoting dimerization via their SH2 domains. The phosphorylated dimer is then actively transported to the nucleus. Once the dimerized STAT protein reaches the nucleus, it binds to a consensus DNA-recognition motif called gamma-activated sites (GAS) in the promoter region of cytokine-inducible genes and activates transcription. The STAT protein can be dephosphorylated by nuclear phosphatases, which leads to inactivation of STAT and subsequent transport out of the nucleus by an exportin-RanGTP complex. There are seven mammalian STAT family members that have been identified: STAT1, STAT2, STAT3, STAT4, STAT5 (STAT5A and STAT5B), and STAT6. STAT1 homodimers are involved in type II interferon signaling, and bind to the GAS (Interferon-Gamma Activated Sequence) promoter to induce expression of ISG (Interferon Stimulated Genes). In type I interferon signaling, a STAT1-STAT2 heterodimer combines with IRF9 (Interferon Response Factor 9) to form ISGF3 (Interferon Stimulated Gene Factor 3), which binds to the ISRE (Interferon-Stimulated Response Element) promoter to induce ISG expression.

Where the engineered cells are T cells, an enhanced immune response may manifest as an increase in the cytolytic response of T cells towards the target cells present in the recipient, e.g. towards elimination of tumor cells and infected cells; a decrease in symptoms of autoimmune disease; and the like.

Where the cells are contacted with the orthogonal ligand in vitro, the cytokine is added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR T cells modified to express an orthogonal chimeric receptor, are infused to the recipient, in combination with or prior to administration of the orthogonal ligand, e.g., IL-2 and allowed to contact cells in their native environment, e.g. in lymph nodes, etc. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., or for systemic administration. Parenteral infusions include intramuscular, intravenous (bolus or slow infusion), intraarterial, intraperitoneal, intrathecal, intratumoral, subcutaneous administration; etc.

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Generally at least about 10⁴ engineered cells/kg are administered, at least about 10⁵ engineered cells/kg; at least about 10⁶ engineered cells/kg, at least about 10⁷ engineered cells/kg, at least about 10⁸ engineered cells/kg, or more. For example, typical ranges for the administration of cells for use in the practice of the present invention range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

For example, in the current clinical practice of CAR T cell therapy, CAR T cells are commonly administered in combination with lymphodepletion (e.g. by administration of Alemtuzumab (monoclonal anti-CD52), purine analogs, and the like) to facilitate expansion of the CAR T cells to prior to host immune recovery. In some embodiments, the CAR T cells may be modified for resistance to alemtuzumab (commercially available under the tradenames Campath® and Lemtrada®). In one aspect of the invention, the lymphodepletion currently employed in association with CAR T therapy may be obviated or reduced by the orthogonal ligand expressing CAR Ts of the present invention. As noted above, the lymphodepletion is commonly employed to enable expansion of the CAR T cells. However, the lymphodepletion is also associated with major side effects of CAR T cell therapy. Because the orthogonal ligand provides a means to selectively expand a particular T cell population, the need for lymphodepletion prior to administration of the orthogonal ligand expressing CAR Ts may be reduced or obviated. The present invention enables the practice of CAR T cell therapy without or with reduced lymphodepletion prior to administration of the orthogonal ligand expressing CAR Ts.

In one embodiment, the present invention provides a method of treating a subject suffering from a disease, disorder or condition amendable to treatment with CAR T cell therapy (e.g. cancer) by the administration of a orthogonal chimeric receptor expressing CAR Ts in the absence of lymphodepletion prior to administration of the orthogonal ligand. In one embodiment, the present invention provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T cells from the individual; (b) enriching the biological sample for the presence of T cells; (c) transfecting the T cells with one or more expression vectors comprising a nucleic acid sequence encoding a CAR and a nucleic acid sequence encoding an orthogonal chimeric receptor, the antigen targeting domain of the CAR being capable of binding to at least one antigen present on the aberrant population of cells; (d) expanding the population of the orthogonal chimeric receptor expressing CAR T cells ex vivo; (e) administering a pharmaceutically effective amount of the orthogonal chimeric receptor expressing CAR T cells to the mammal; and (f) modulating the growth of the orthogonal chimeric receptor expressing CAR T cells using an ligand that binds selectively to the orthogonal chimeric receptor expressed on the CAR T cell. In one embodiment, the foregoing method is associated with lymphodepletion or immunosuppression of the mammal prior to the initiation of the course of CAR T cell therapy. In another embodiment, the foregoing method is practiced in the absence of lymphodepletion and/or immunosuppression of the mammal.

The preferred formulation depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Also provided are kits for use in the methods. The subject kits include an expression vector encoding an orthogonal chimeric receptor, or a cell comprising the expression vector. Kits may further comprise the cognate orthogonal ligand. In some embodiments, the components are provided in a dosage form (e.g., a therapeutically effective dosage form), in liquid or solid form in any convenient packaging (e.g., stick pack, dose pack, etc.). Reagents for the selection or in vitro derivation of cells may also be provided, e.g. growth factors, differentiation agents, tissue culture reagents; and the like.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Therapeutic Methods

In some embodiments the subject compositions, methods and kits are used to enhance a T cell mediated immune response. In some embodiments the immune response is directed towards a condition where it is desirable to deplete or regulate target cells, e.g., cancer cells, infected cells, regulation of immune cells, including without limitation immune cells involved in autoimmune disease, immune cells involved in transplantation, undesirable inflammatory responses, enhancing erythropoiesis, enhancing thrombopoiesis, etc. Immune conditions may include, without limitation, autoimmune diseases, graft v host disease, hematopoietic bone marrow transplantation, adoptive cell therapy, tumor infiltrating cell (TIL) therapy, inflammation, graft rejection, and the like.

In some embodiments the condition is cancer. As used herein, the terms “cancer” (or “cancerous”), “hyperproliferative,” and “neoplastic” to refer to cells having the capacity for autonomous or unregulated growth (e.g., an abnormal state or condition characterized by rapidly proliferating cell growth). Hyperproliferative and neoplastic disease states may be categorized as pathologic (e.g., characterizing or constituting a disease state), or they may be categorized as non- pathologic (e.g., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures.

Examples of tumor cells include but are not limited to AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. Any cancer, where the cancer cells exhibit increased expression of CD47 compared to non-cancer cells, is a suitable cancer to be treated by the subject methods and compositions.

The compositions and method of the present invention may be combined with additional therapeutic agents. For example, when the disease, disorder or condition to be treated is a neoplastic disease (e.g. cancer) the methods may be combined with conventional chemotherapeutic agents or other biological anti-cancer drugs such as checkpoint inhibitors (e.g. PD1 or PDL1 inhibitors) or therapeutic monoclonal antibodies (e.g., Avastin®, Herceptin®).

Examples of chemical agents identified in the art as useful in the treatment of neoplastic disease, include without limitation, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Targeted therapeutics that can be administered in combination may include, without limitation, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade), Jakafi (ruxolitinib); Janus kinase inhibitors, such as tofacitinib; ALK inhibitors, such as crizotinib; Bcl-2 inhibitors, such as obatoclax, venclexta, and gossypol; FLT3 inhibitors, such as midostaurin (Rydapt), IDH inhibitors, such as AG-221, PARP inhibitors, such as Iniparib and Olaparib; P13K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

Examples of biological agents identified in the art as useful in the treatment of neoplastic disease, include without limitation, cytokines or cytokine antagonists such as IL-12, INFa, or anti-epidermal growth factor receptor, radiotherapy, irinotecan; tetrahydrofolate antimetabolites such as pemetrexed; antibodies against tumor antigens, a complex of a monoclonal antibody and toxin, a T cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens readily appreciated by the skilled clinician in the art.

Tumor specific monoclonal antibodies that can be administered in combination with an engineered cell may include, without limitation, Rituximab (marketed as MabThera® or Rituxan®), Alemtuzumab, Panitumumab, Ipilimumab (Yervoy®), etc.

In some embodiments the compositions and methods of the present invention may be combined with immune checkpoint therapy. Examples of immune checkpoint therapies include inhibitors of the binding of PD1 to PDL1 and/or PDL2. PD1 to PDL1 and/or PDL2 inhibitors are well known in the art. Examples of commercially available monoclonal antibodies that interfere with the binding of PD1 to PDL1 and/or PDL2 include nivolumab (Opdivo®, BMS-936558, MDX1106, commercially available from BristolMyers Squibb, Princeton N.J.), pembrolizumab (Keytruda®MK-3475, lambrolizumab, commercially available from Merck and Company, Kenilworth N.J.), and atezolizumab (Tecentriq®, Genentech/Roche, South San Francisco Calif.). Additional examples of PD1 inhibitory antibodies include but are not limited to durvalumab (MED14736, Medimmune/AstraZeneca), pidilizumab (CT-011, CureTech), PDR001 (Novartis), BMS-936559 (MDX1105, Bristol Myers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011. Additionally, small molecule PD1 to PDL1 and/or PDL2 inhibitors are known in the art. See, e.g. Sasikumar, et al as WO2016142833A1 and Sasikumar, et al. WO2016142886A2, BMS-1166 and BMS-1001 (Skalniak, et al (2017) Oncotarget 8(42): 72167-72181).

In other embodiments the methods of the invention are used in the treatment of infection. As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) that induces increased CD47 expression in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

Treatment may be combined with other active agents. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor a, interleukin 12, etc. Antiviral agents, e.g. acyclovir, gancyclovir, etc., may also be used in treatment.

In yet other embodiments, regulatory T cells are engineered for the treatment of autoimmune disease. The spectrum of inflammatory diseases and diseases associated with inflammation is broad and includes autoimmune diseases such rheumatoid arthritis (RA), systemic lupus erythematosus (SLE), multiple sclerosis (MS), and autoimmune hepatitis; insulin dependent diabetes mellitus, degenerative diseases such as osteoarthritis (OA), Alzheimer's disease (AD), and macular degeneration.

Many, if not most, autoimmune and inflammatory diseases involve multiple types of T cells, e.g. TH1, TH2, TH17, and the like. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

The invention now being fully described, it will be apparent to one of ordinary skill in the art that various changes and modifications can be made without departing from the spirit or scope of the invention.

### EXAMPLE 1

### Materials and Methods

Protein production. orthoIL2 was cloned into the insect expression vector pAcGP67-A, and expressed in Trichoplusia ni (High FiveT™) cells (Invitrogen) using the BaculoGold™ baculovirus expression system (BD Biosciences) as previously described (Sockolosky et. al. Science (2018) 359(6379):1037-1042).

Mammalian expression vectors. cDNA encoding full-length mouse orthoRb and orthoRb-ICD chimeric receptors were PCR cloned into pMSCV-IRES-YFP retroviral vector.

Cell culture and Retrovirus production. HEK293T cells were maintained in DMEM supplemented with 10% Fetal Bovine Serum (FBS), 1% L-glutamine (L-glu), and 1% penicillin/streptomycin (P/S). To produce retrovirus, HEK293T cells were transfected with pMSCV retroviral vector and pCL-Eco packaging vector at ratio of 1.5:1 using X-tremeGene™ HP (Roche). 24 h post transfection, media was removed and replenished with DMEM containing 5% FBS and cultured for an additional 24 h. Media was collected (RV supe), clarified using a 0.45 μm filter, and flash frozen in liquid nitrogen for storage at −80 C. Media was replenished (DMEM/5% FBS) and cells were cultured for an additional 24 h and virus was collected and stored as above.

Isolation and retroviral transduction of primary mouse T cells. Cells from the spleen and lymph nodes of C57BL/6J mice were harvested, processed to a single cell suspension, and activated on plate-bound anti-CD3 (145-2C11, 2.5 μg/ml) and soluble anti-CD28 (37.51, 5 ug/ml) in T cell media (RPMI-1640, 10% FBS, HEPES, 1% Pen/Strep, Glutamax, β-mercaptoethanol, Sodium pyruvate, and NEAA) supplemented with 100 IU/ml mIL2. 24 h post activation, cells were resuspended in viral supernatant (RV supe) containing polybrene and 100 IU/m1 mIL2, and spinfected at 2700 rpm, 32 C for 90′. RV supe was then removed, and cells replenished with T cell media containing mIL2. 24 h post transduction, cells were harvested and expanded in T cell media containing mIL2 for 24 h. Media was then exchanged, and cells allowed to rest in T cell media lacking mIL2 for an additional 24 h before being used for in vitro signaling or proliferation assays.

In Vitro phospho-signaling assay. RV transduced activated/rested primary mouse T cells were plated at 1×10⁵ cells per well in ultra-low binding 96-well round bottom plate (Cat. 7007; Costar) in 100 μL warm media. Cells were stimulated by addition of 100 μL solution of serial dilutions of o182 for 20′ at 37° C. and the reaction was terminated by 1.5% paraformaldehyde (PFA) fixation for 10′ at RT with agitation. Cells were then permeabilized with 100% ice-cold methanol for at least 45′ on ice or stored at −80 ° C. overnight. Fixed, permeabilized cells were washed 3× with FACS buffer and intracellular phosphorylated STAT proteins were detected with anti-STATS pY694-Alexa647, anti-STAT3 pY705-Alexa647 (BD Biosciences), or anti-STAT1 pY701-Alexa647 (Cell Signaling) diluted 1:100 in FACS buffer and incubated for 1 h at 4 C. Cells were washed and analyzed on a CytoFLEX equipped with a high-throughput autosampler (Beckman Coulter). Data represent the mean fluorescence intensity (MFI) and points were fit to a sigmoidal dose response curve using Prism 8 (GraphPad). All data are presented as mean (n=2/3)±SEM.

In vitro primary mouse T cell proliferation assay. RV transduced activated/rested primary mouse T cells were labeled with CellTracer Violet according to manufacturer protocol (Molecular Probes), and cultured at 1×10⁵ cells per well in ultra-low binding 96-well round bottom with serial dilutions of o182. 72 h later, cells were analyzed on the CytoFLEX (Beckman Coulter).

Animals. C57BL/6J (Cat. 000664) mice were purchased from Jackson Labs, and housed at Stanford University Animal Facility according to approved protocols.

### Results

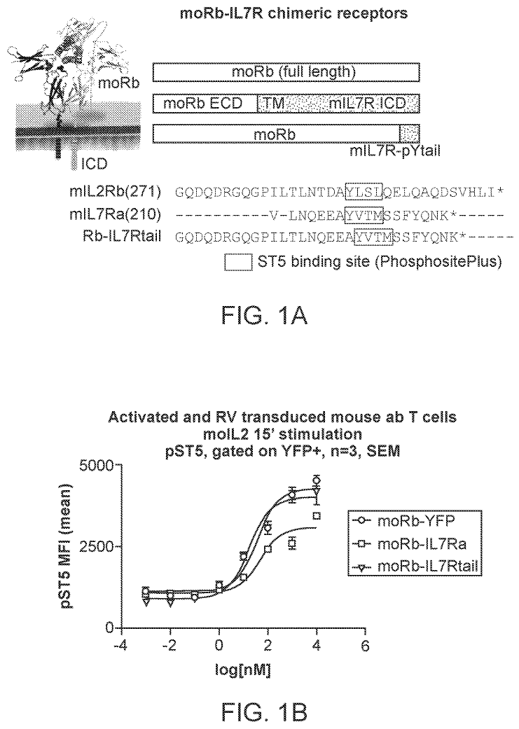

As shown in FIG. 1A, chimeric proteins were designed. Murine ortho IL-2Rp (mIL2Rb) chimeric proteins include a chimera comprising the extracellular domain of moRb and the transmembrane and intracellular domains of murine IL-7 receptor (SEQ ID NO:4), and a chimera of comprising the extracellular, transmembrane and partial intracellular domains of the murine ortho IL-2Rβ and the IL-7 receptor tail (SEQ ID NO:6). The C-termini with STAT5 signaling protein binding site includes a tyrosine target residue (pY) for phosphorylation.

T cells isolated from BL6 mice and activated by contacting with anti-CD3/anti-CD28 coated beads and, and transduced with recombinant retroviral vectors encoding the indicated chimeric proteins, the retroviral construct containing an IRES sequence and yellow fluorescent protein (YFP). Transduced cells were stimulated with mouse ortho-IL2 (SEQ ID NO:30) for 15 minutes, then fixed in paraformaldehyde (PFA), methanol (MeOH) permeabilized and stained with anti-pSTAT5-A647 antibody. Samples were analyzed on a CytoFLEX® flow cytometer (Beckman Coulter Life Sciences, Indianapolis IN) gating on YFP+ cell data plotted with Prism® software (GraphPad Software, San Diego Calif. USA). SEM, n=3. The data shown in FIG. 1B demonstrate a change in the phosphorylation of STAT5, which varies according to the intracellular domain of the receptor.

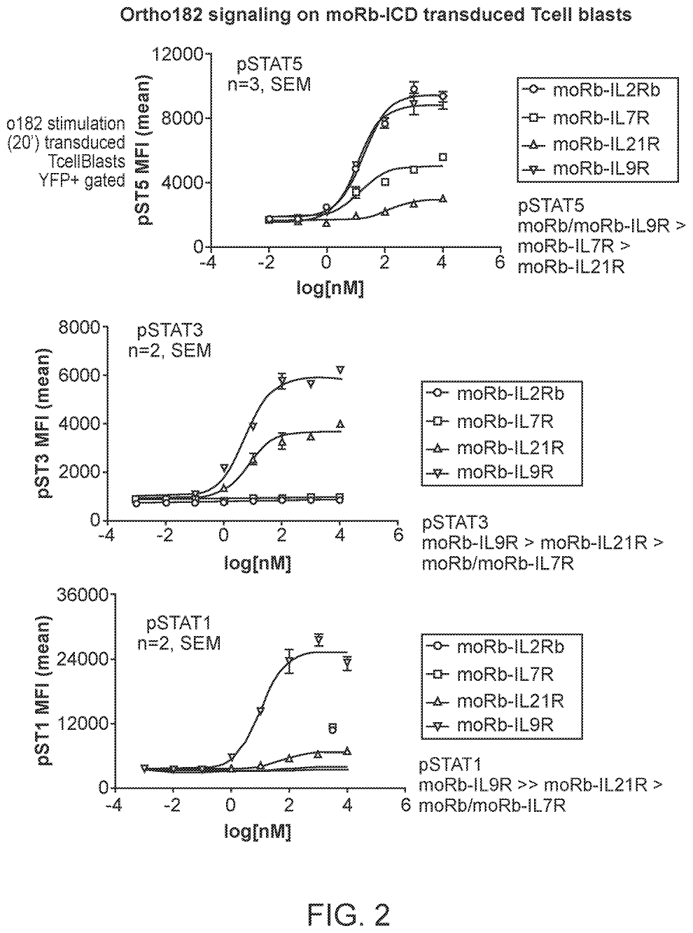

STAT5, STAT3 and STAT1 signaling in T cell blasts recombinantly modified to express chimeric fusion receptors comprising the ortho IL2 extracellular domains and the transmembrane and intracellular signaling domains of the IL2 receptor beta subunit (moRb-IL2Rb), IL7 receptor (moRb-IL7), IL21 receptor (moRb-IL21) and IL9 receptor (moRb-IL9) were evaluated in response to exposure to the ortho-IL2 ligand. T cells from BL6 mice were isolated, anti-CD3/anti-CD28 activated and transduced with the indicated moRb IRES YFP retrovirus (RV): moRb (SEQ ID NO:2), moRb-IL-7R (SEQ ID NO:4), moRb-IL21R (SEQ ID NO:10), mRb-IL-9R (SEQ ID NO:8). Transduced cells were stimulated with ortho IL2 (SEQ ID NO:30) for 20′, then fixed in PFA, MeOH permeabilized and stained with anti-pSTAT5-A647 antibody, anti-pSTAT3-A647 antibody, or anti-pSTAT1-A647 antibody. The samples were analyzed on a CytoFLEX® flow cytometer, gating on YFP+ cells and the data plotted with the assistance of the Prism® software. The data show that the fusion receptors provide phosphorylation of STAT1, 3 and 5 intracellular signaling characteristic of the phosphorylation pattern characteristic of the receptor from which the intracellular domain was derived while maintaining the same IL-2 orthogonal extracellular receptor domain. The data is shown in FIG. 2.

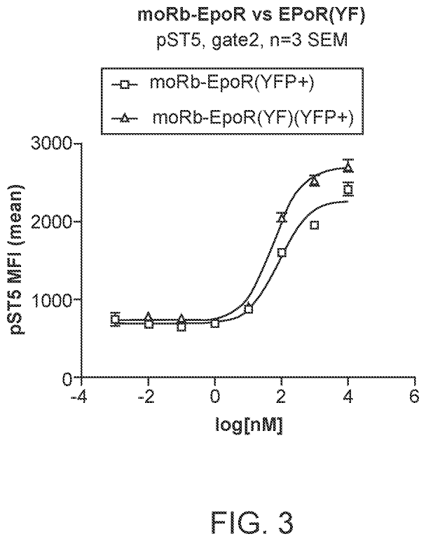

T cell blasts transduced with a vector encoding chimeric receptor comprising the extracellular domain of murine ortho IL-1 and the transmembrane and intracellular signaling domains of the erythropoietin (EPO) receptor (oRb-EpoR) were stimulated with ortho-IL2, demonstrating that the fusion receptor is capable of intracellular signaling and activating pSTAT5, a signal characteristic of an activated EPO receptor. Briefly, T cells from BL6 mice were isolated, anti-CD3/anti-CD28 activated and transduced with indicated with retroviral expression vectors comprising a IRES bi-cistronic expression cassette, the first cistrons comprising nucleic acid sequence encoding the moRb-EpoR fusion receptor (SEQ ID NO:12) or moRb-EpoR-YF fusion receptor (SEQ ID NO:14) with, in each case, the second cistron comprising a nucleic acid sequence encoding YFP. Transduced cells were stimulated with ortho IL2 for 20 minutes, then fixed in PFA, MeOH permeabilized and stained with anti-pSTAT5-A647. The samples were analyzed on a CytoFLEX® flow cytometer, gating on YFP+ cells and the data plotted with the assistance of the Prism® software. The data provided in FIG. 3 show that STAT5 phosphorylation, a signal characteristic of the EPO receptor increases after ortho-IL2 stimulation of the ECD of the fusion receptor.

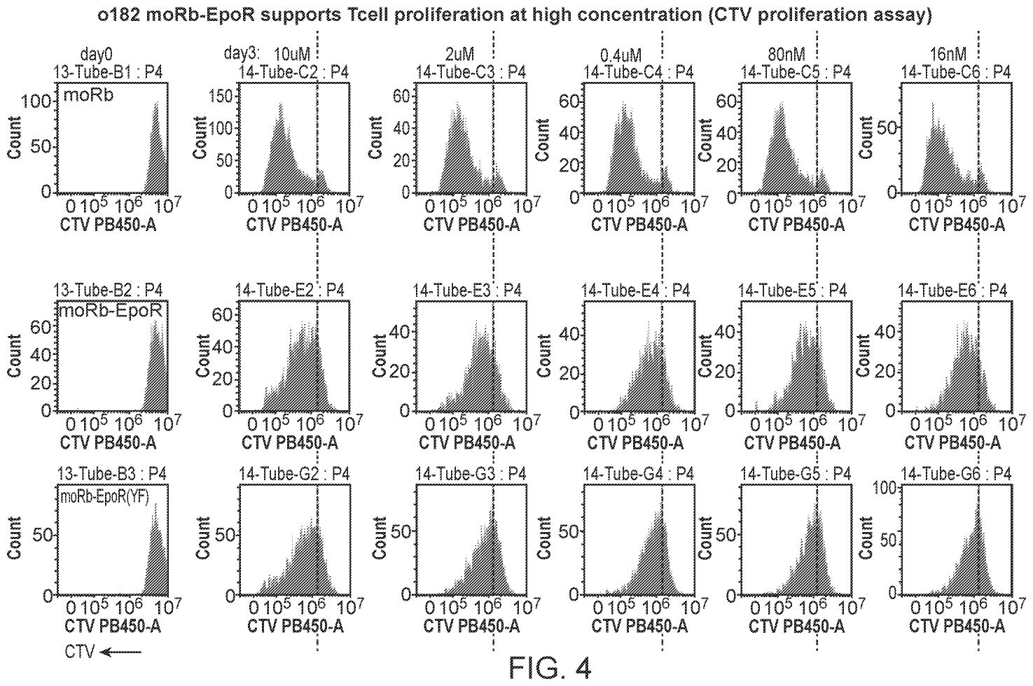

Data were generated to demonstrate that ortho-IL-2 induces proliferation in T cells transduced with a recombinant retroviral encoding chimeric receptors. Briefly, T cells from BL6 mice were isolated, anti-CD3/anti-CD28 activated and transduced with indicated retrovirus: moRb (SEQ ID NO:2), moRb-EpoR (SEQ ID NO:12) or moRb-EpoR(YF) (SEQ ID NO:14). Cells were labeled with CellTraceTM Violet (CTV, Thermo Fisher Scientific) on day 0, and incubated with indicated concentration of ortho-IL2 (SEQ ID NO:30). On day 3, samples were analyzed on a CytoFLEX® flow cytomer, gating on live, YFP+ cells. FIG. 4 provides representative data from 4 replicates of the experiment. The data demonstrate an ortho-IL2 dose dependent increase proliferation of T cells.

### PROTEIN SEQUENCES REFERENCED

The present disclosure makes reference to the following protein sequences:

A. Mouse ortho-IL-2Rβ Receptor sequences:
1. SEQ ID NO: 1: Mouse orthoIL2Rβ (moRb) coding sequence:
(SEQ ID NO: 1)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCAGATCCCATGAAGGAGATCCTCCCCATGTCATGGCTCAGATACCTTCTGCTGGTCCTTGGTTGTTT
TTCTGGCTTCTTCTCCTGCGTCTACATTTTGGTCAAGTGCCGGTACCTTGGGCCATGGCTGAAGACAGTTCTCAAGT
GCCACATCCCAGATCCTTCTGAGTTCTTCTCCCAGCTGAGCTCCCAGCATGGGGGAGACCTTCAGAAATGGCTCTCC
TCGCCTGTCCCCTTGTCCTTCTTCAGCCCCAGTGGCCCTGCCCCTGAGATCTCTCCGCTGGAAGTGCTCGACGGAGA
TTCCAAGGCCGTGCAGCTGCTCCTGTTACAGAAGGACTCTGCCCCTTTACCCTCGCCCAGCGGCCACTCACAGGCCA
GCTGCTTCACCAACCAGGGCTACTTCTTCTTCCATCTGCCCAATGCCTTGGAGATCGAATCCTGCCAGGTGTACTTC
ACCTATGACCCCTGTGTGGAAGAGGAGGTGGAGGAGGATGGGTCAAGGCTGCCCGAGGGATCTCCCCACCCACCTCT
GCTGCCTCTGGCTGGAGAACAGGATGACTACTGTGCCTTCCCGCCCAGGGATGACCTGCTGCTCTTCTCCCCGAGCC
TCAGCACCCCCAACACTGCCTATGGGGGCAGCAGAGCCCCTGAAGAAAGATCTCCACTCTCCCTGCATGAGGGACTT
CCCTCCCTAGCATCCCGTGACCTGATGGGCTTACAGCGCCCTCTGGAGCGGATGCCGGAAGGTGATGGAGAGGGGCT
GTCTGCCAATAGCTCTGGGGAGCAGGCCAGTGTCCCAGAAGGCAACCTTCATGGGCAAGATCAGGACAGAGGCCAGG
GCCCCATCCTGACCCTGAACACCGATGCCTATCTGTCTCTTCAAGAACTACAGGCCCAAGATTCAGTCCACCTAATA
TGA
2. SEQ ID NO: 2 -- Mouse ortho IL-2R□ protein sequence:
(SEQ ID NO: 2)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPADPMKEILPMSWLRYLLLVLGCFSGFFSCVYILVKCRYLGPWLKTVLKCHIPDPSEFFSQLSSQHGGDLQKWLS
SPVPLSFFSPSGPAPEISPLEVLDGDSKAVQLLLLQKDSAPLPSPSGHSQASCFTNQGYFFFHLPNALEIESCQVYF
TYDPCVEEEVEEDGSRLPEGSPHPPLLPLAGEQDDYCAFPPRDDLLLFSPSLSTPNTAYGGSRAPEERSPLSLHEGL
PSLASRDLMGLQRPLERMPEGDGEGLSANSSGEQASVPEGNLHGQDQDRGQGPILTLNTDAYLSLQELQAQDSVHLI
The above mouse ortho IL-2Rβ orthogonal receptor (SEQ ID NO. 2) is derived from
the wild-type murine IL-2Rβ receptor but contains amino acid substitutions
H134D and Y135F relative to the wild type murine IL2Rβ protein.
3. SEQ ID NO: 3 mouse ortho IL-2Rβ/IL-7 receptor chimera coding sequence
(SEQ ID NO: 3)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCAAAGAATCAAGGAGGATGGGATCCTGTCTTGCCAAGTGTCACCATTCTGAGTTTGTTCTCTGTGTT
TTTGTTGGTCATCTTAGCCCATGTGCTATGGAAAAAAAGGATTAAACCTGTCGTATGGCCTAGTCTCCCCGATCATA
AGAAAACTCTGGAACAACTATGTAAGAAGCCAAAAACGAGTCTGAATGTGAGTTTCAATCCCGAAAGTTTCCTGGAC
TGCCAGATTCATGAGGTGAAAGGCGTTGAAGCCAGGGACGAGGTGGAAAGTTTTCTGCCCAATGATCTTCCTGCACA
GCCAGAGGAGTTGGAGACACAGGGACACAGAGCCGCTGTACACAGTGCAAACCGCTCGCCTGAGACTTCAGTCAGCC
CACCAGAAACAGTTAGAAGAGAGTCACCCTTAAGATGCCTGGCTAGAAATCTGAGTACCTGCAATGCCCCTCCACTC
CTTTCCTCTAGGTCCCCTGACTACAGAGATGGTGACAGAAATAGGCCTCCTGTGTATCAAGACTTGCTGCCAAACTC
TGGAAACACAAATGTCCCTGTCCCTGTCCCTCAACCATTGCCTTTCCAGTCGGGAATCCTGATACCAGTTTCTCAGA
GACAGCCCATCTCCACTTCCTCAGTACTGAATCAAGAAGAAGCGTATGTCACCATGTCTAGTTTTTACCAAAACAAA
TGA
4. SEQ ID NO: 4 mouse ortho IL-2Rβ/IL-7 receptor chimera Protein sequence:
(SEQ ID NO: 4)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPAKNQGGWDPVLPSVTILSLFSVFLLVILAHVLWKKRIKPVVWPSLPDHKKTLEQLCKKPKTSLNVSFNPESFLD
CQIHEVKGVEARDEVESFLPNDLPAQPEELETQGHRAAVHSANRSPETSVSPPETVRRESPLRCLARNLSTCNAPPL
LSSRSPDYRDGDRNRPPVYQDLLPNSGNTNVPVPVPQPLPFQSGILIPVSQRQPISTSSVLNQEEAYVTMSSFYQNK
Residues 1-235 of the IL-2Rβ/IL-7 orthogonal chimeric receptor (SEQ ID NO: 4)
are derived from ortho IL-2Rβ (SEQ ID NO: 2) and residues 236-462 (underlined)
are obtained from the murine IL-7R protein.
5. SEQ ID NO: 5 Mouse orthoIL2Rb-IL7Rtail (moRb-IL7Rtail) coding sequence
(SEQ ID NO: 5)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCAGATCCCATGAAGGAGATCCTCCCCATGTCATGGCTCAGATACCTTCTGCTGGTCCTTGGTTGTTT
TTCTGGCTTCTTCTCCTGCGTCTACATTTTGGTCAAGTGCCGGTACCTTGGGCCATGGCTGAAGACAGTTCTCAAGT
GCCACATCCCAGATCCTTCTGAGTTCTTCTCCCAGCTGAGCTCCCAGCATGGGGGAGACCTTCAGAAATGGCTCTCC
TCGCCTGTCCCCTTGTCCTTCTTCAGCCCCAGTGGCCCTGCCCCTGAGATCTCTCCGCTGGAAGTGCTCGACGGAGA
TTCCAAGGCCGTGCAGCTGCTCCTGTTACAGAAGGACTCTGCCCCTTTACCCTCGCCCAGCGGCCACTCACAGGCCA
GCTGCTTCACCAACCAGGGCTACTTCTTCTTCCATCTGCCCAATGCCTTGGAGATCGAATCCTGCCAGGTGTACTTC
ACCTATGACCCCTGTGTGGAAGAGGAGGTGGAGGAGGATGGGTCAAGGCTGCCCGAGGGATCTCCCCACCCACCTCT
GCTGCCTCTGGCTGGAGAACAGGATGACTACTGTGCCTTCCCGCCCAGGGATGACCTGCTGCTCTTCTCCCCGAGCC
TCAGCACCCCCAACACTGCCTATGGGGGCAGCAGAGCCCCTGAAGAAAGATCTCCACTCTCCCTGCATGAGGGACTT
CCCTCCCTAGCATCCCGTGACCTGATGGGCTTACAGCGCCCTCTGGAGCGGATGCCGGAAGGTGATGGAGAGGGGCT
GTCTGCCAATAGCTCTGGGGAGCAGGCCAGTGTCCCAGAAGGCAACCTTCATGGGCAAGATCAGGACAGAGGCCAGG
GCCCCATCCTGACCCTGAATCAAGAAGAAGCGTATGTCACCATGTCTAGTTTTTACCAAAACAAATGA
6. SEQ ID NO: 6 Mouse orthoIL2Rb-IL7Rtail (moRb-IL7Rtail) protein sequence:
(SEQ ID NO: 6)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPADPMKEILPMSWLRYLLLVLGCFSGFFSCVYILVKCRYLGPWLKTVLKCHIPDPSEFFSQLSSQHGGDLQKWLS
SPVPLSFFSPSGPAPEISPLEVLDGDSKAVQLLLLQKDSAPLPSPSGHSQASCFTNQGYFFFHLPNALEIESCQVYF
TYDPCVEEEVEEDGSRLPEGSPHPPLLPLAGEQDDYCAFPPRDDLLLFSPSLSTPNTAYGGSRAPEERSPLSLHEGL
PSLASRDLMGLQRPLERMPEGDGEGLSANSSGEQASVPEGNLHGQDQDRGQGPILTLNQEEAYVTMSSFYQNK
Residues 1-520 of the moRb-IL7Rtail chimeric orthogonal receptor (SEQ ID NO:
6) are derived from ortho IL-2Rβ (SEQ ID NO: 2) and residues 521-535 (underlined)
of the moRb-IL7Rtail (SEQ ID NO: 6) are derived from the mouse IL-7R protein.
7. SEQ ID NO: 7 Mouse ortho IL2Rβ-IL9R chimera(moRb-IL9R) coding sequence
(SEQ ID NO: 7)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCACAGAGGAGACAGGGCCTCCTGGTCCCACGCTGGCAATGGTCAGCCAGCATCCTTGTAGTTGTGCC
CATCTTTCTTCTGCTGACTGGCTTTGTCCACCTTCTGTTCAAGCTGTCACCCAGGCTGAAGAGAATCTTTTACCAGA
ACATTCCATCTCCCGAGGCGTTCTTCCATCCTCTCTACAGTGTGTACCATGGGGACTTCCAGAGTTGGACAGGGGCC
CGCAGAGCCGGACCACAAGCAAGACAGAATGGTGTCAGTACTTCATCAGCAGGCTCAGAGTCCAGCATCTGGGAGGC
CGTCGCCACACTCACCTATAGCCCGGCATGCCCTGTGCAGTTTGCCTGCCTGAAGTGGGAGGCCACAGCCCCGGGCT
TCCCAGGGCTCCCAGGCTCAGAGCATGTGCTGCCGGCAGGGTGTCTGGAGTTGGAAGGACAGCCATCTGCCTACCTG
CCCCAGGAGGACTGGGCCCCACTGGGCTCTGCCAGGCCCCCTCCTCCAGACTCAGACAGCGGCAGCAGCGACTATTG
CATGTTGGACTGCTGTGAGGAATGCCACCTCTCAGCCTTCCCAGGACACACCGAGAGTCCTGAGCTCACGCTAGCTC
AGCCTGTGGCCCTTCCTGTGTCCAGCAGGGCCTGA
8. SEQ ID NO: 8 Mouse orthoIL2Rβ-IL9R chimera(moRb-IL9R) protein sequence:
(SEQ ID NO: 8)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPAQRRQGLLVPRWQWSASILVVVPIFLLLTGFVHLLFKLSPRLKRIFYQNIPSPEAFFHPLYSVYHGDFQSWTGA
RRAGPQARQNGVSTSSAGSESSIWEAVATLTYSPACPVQFACLKWEATAPGFPGLPGSEHVLPAGCLELEGQPSAYL
PQEDWAPLGSARPPPPDSDSGSSDYCMLDCCEECHLSAFPGHTESPELTLAQPVALPVSSRA
Residues 1-235 of moRb-IL9R chimeric orthogonal receptor (SEQ ID NO: 8) are
derived from the ortho IL-2Rβ (SEQ ID NO: 2) and residues 236-447 (underlined)
of moRb-IL9R (SEQ ID NO: 8) are derived from the mouse IL-9R.
9. SEQ ID NO: 9 Mouse orthoIL2Rβ-IL21R chimera (moRb-IL21R) coding sequence
(SEQ ID NO: 9)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGACCTGCTGGCGAACCTGAAGCTGGATGGGACCCTCATATGTTGCTGCTGCTGGCCGTGCTGATCATCGTGCT
GGTGTTCATGGGCCTGAAGATCCATCTGCCTTGGAGACTGTGGAAGAAAATCTGGGCCCCTGTGCCTACTCCTGAGA
GCTTCTTCCAGCCACTGTACAGAGAGCACAGCGGCAACTTCAAGAAATGGGTCAACACCCCTTTCACCGCCAGCAGT
ATCGAGCTGGTGCCTCAGAGCAGCACCACAACATCTGCCCTGCACCTGTCTCTGTACCCCGCCAAAGAGAAGAAGTT
CCCTGGCCTGCCTGGACTGGAAGAACAGCTGGAATGTGACGGCATGAGCGAGCCTGGCCACTGGTGTATCATTCCTC
TGGCTGCTGGACAGGCCGTGTCCGCCTATAGCGAGGAAAGAGACAGACCCTACGGCCTGGTGTCCATCGACACAGTG
ACAGTGGGAGATGCCGAGGGCCTGTGTGTGTGGCCTTGTAGCTGTGAAGATGACGGCTACCCTGCCATGAACCTGGA
TGCCGGAAGAGAGAGCGGCCCTAACTCTGAGGATCTGCTGCTCGTGACCGATCCTGCCTTCCTGTCTTGCGGCTGTG
TGTCTGGATCTGGCCTGAGACTCGGAGGCTCTCCTGGAAGCCTGCTGGATAGACTGAGACTGAGCTTCGCCAAAGAA
GGCGACTGGACCGCCGATCCTACTTGGAGAACAGGATCTCCTGGCGGCGGAAGCGAATCTGAAGCAGGTTCTCCACC
TGGCCTGGACATGGACACATTCGACTCTGGCTTCGCCGGCAGCGATTGTGGAAGCCCTGTGGAAACAGACGAGGGCC
CACCTAGAAGCTACCTGAGACAGTGGGTCGTGCGGACACCTCCTCCAGTTGATTCTGGCGCCCAGTCCTCTTGA
10. SEQ ID NO: 10 Mouse orthoIL2Rβ-IL21R chimera (moRb-IL21R) protein sequence:
(SEQ ID NO: 10)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPAGEPEAGWDPHMLLLLAVLIIVLVFMGLKIHLPWRLWKKIWAPVPTPESFFQPLYREHSGNFKKWVNTPFTASS
IELVPQSSTTTSALHLSLYPAKEKKFPGLPGLEEQLECDGMSEPGHWCIIPLAAGQAVSAYSEERDRPYGLVSIDTV
TVGDAEGLCVWPCSCEDDGYPAMNLDAGRESGPNSEDLLLVTDPAFLSCGCVSGSGLRLGGSPGSLLDRLRLSFAKE
GDWTADPTWRTGSPGGGSESEAGSPPGLDMDTFDSGFAGSDCGSPVETDEGPPRSYLRQWVVRTPPPVDSGAQSS
Residues 1-235 of moRb-IL21R chimeric orthogonal receptor (SEQ ID NO: 10) are
derived from ortho IL-2Rβ (SEQ ID NO. 2) and residues 236-537 (underlined) of
moRb-IL21R chimeric orthogonal recpetor are derived from the mouse IL-21R.
11. SEQ ID NO: 11 Mouse orthoIL2Rβ-EpoR (moRb-EpoR) coding sequence:
(SEQ ID NO: 11)
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCAAGCGATCTGGACCCTCTGATCCTGACACTGAGCCTGATCCTGGTGCTGATCTCCCTGCTGCTGAC
AGTGCTGGCCCTGCTGAGCCACAGAAGAACCCTGCAGCAGAAGATCTGGCCTGGCATCCCATCTCCAGAGAGCGAGT
TCGAGGGCCTGTTCACCACACACAAGGGCAACTTCCAGCTGTGGCTGCTGCAGCGAGATGGCTGTCTTTGGTGGTCC
CCTGGCAGCAGCTTTCCTGAGGATCCACCAGCTCACCTGGAAGTGCTGAGCGAGCCTAGATGGGCTGTTACACAGGC
TGGCGATCCTGGCGCCGATGATGAAGGACCTCTGCTGGAACCTGTGGGCTCTGAACATGCCCAGGACACCTATCTGG
TGCTGGACAAGTGGCTGCTCCCCAGAACACCCTGTAGCGAGAATCTGTCTGGCCCTGGCGGATCCGTGGATCCCGTG
ACAATGGATGAGGCCAGCGAGACAAGCAGCTGCCCTTCTGATCTGGCCAGCAAGCCTAGACCTGAGGGCACAAGCCC
TAGCAGCTTCGAGTACACCATTCTGGACCCCAGCAGCCAGCTGCTGTGTCCTAGAGCACTGCCTCCAGAGCTGCCTC
CTACACCTCCTCACCTGAAGTACCTGTACCTGGTGGTGTCCGACAGCGGCATCAGCACCGATTATAGCTCTGGTGGC
TCTCAGGGCGTGCACGGCGATAGTTCTGATGGCCCTTACTCTCACCCCTACGAAAACAGCCTGGTGCCTGACAGCGA
ACCTCTGCACCCTGGATACGTGGCCTGTAGCTAA
12. SEQ ID NO: 12 Mouse orthoIL2Rβ-EpoR (moRb-EpoR) protein sequence:
(SEQ ID NO: 11)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPASDLDPLILTLSLILVLISLLLTVLALLSHRRTLQQKIWPGIPSPESEFEGLFTTHKGNFQLWLLQRDGCLWWS
PGSSFPEDPPAHLEVLSEPRWAVTQAGDPGADDEGPLLEPVGSEHAQDTYLVLDKWLLPRTPCSENLSGPGGSVDPV
TMDEASETSSCPSDLASKPRPEGTSPSSFEYTILDPSSQLLCPRALPPELPPTPPHLKYLYLVVSDSGISTDYSSGG
SQGVHGDSSDGPYSHPYENSLVPDSEPLHPGYVACS
Residues 1-235 of the moRb-EpoR chimeric orthogonal receptor (SEQ ID NO: 11) are
derived from ortho IL-2Rβ (SEQ ID NO. 2) and residues 236-498 (underlined) of
moRb-EpoR (SEQ ID NO: 11) are derived from mouse EpoR.
13. SEQ ID NO: 13 Mouse orthoIL2Rb-EpoR(ITIM YF) (moRb-EpoR(YF) coding sequence
ATGGCTACCATAGCTCTTCCCTGGAGCCTGTCCCTCTACGTCTTCCTCCTGCTCCTGGCTACACCTTGGGCATCTGC
AGCAGTGAAAAACTGTTCCCATCTTGAATGCTTCTACAACTCAAGAGCCAATGTCTCTTGCATGTGGAGCCATGAAG
AGGCTCTGAATGTCACAACCTGCCACGTCCATGCCAAGTCGAACCTGCGACACTGGAACAAAACCTGTGAGCTAACT
CTTGTGAGGCAGGCATCCTGGGCCTGCAACCTGATCCTCGGGTCGTTCCCAGAGTCCCAGTCACTGACCTCCGTGGA
CCTCCTTGACATAAATGTGGTGTGCTGGGAAGAGAAGGGTTGGCGTAGGGTAAAGACCTGCGACTTCCATCCCTTTG
ACAACCTTCGCCTGGTGGCCCCTCATTCCCTCCAAGTTCTGCACATTGATACCCAGAGATGTAACATAAGCTGGAAG
GTCTCCCAGGTCTCTGACTTCATTGAACCATACTTGGAATTTGAGGCCCGTAGACGTCTTCTGGGCCACAGCTGGGA
GGATGCATCCGTATTAAGCCTCAAGCAGAGACAGCAGTGGCTCTTCTTGGAGATGCTGATCCCTAGTACCTCATATG
AGGTCCAGGTGAGGGTCAAAGCTCAACGAAACAATACCGGGACCTGGAGTCCCTGGAGCCAGCCCCTGACCTTTCGG
ACAAGGCCAGCAAGCGATCTGGACCCTCTGATCCTGACACTGAGCCTGATCCTGGTGCTGATCTCCCTGCTGCTGAC
AGTGCTGGCTCTGCTGAGCCACAGAAGAACCCTGCAGCAGAAGATCTGGCCTGGCATCCCATCTCCAGAGAGCGAGT
TCGAGGGCCTGTTCACCACACACAAGGGCAACTTCCAGCTGTGGCTGCTGCAGCGAGATGGCTGTCTTTGGTGGTCC
CCTGGCTCTAGCTTTCCTGAGGACCCTCCTGCTCACCTGGAAGTGCTGTCTGAGCCTAGATGGGCCGTTACACAGGC
TGGCGATCCAGGCGCTGATGATGAAGGACCTCTGCTGGAACCTGTGGGCTCTGAGCACGCTCAGGACACCTATCTGG
TGCTGGACAAGTGGCTGCTCCCCAGAACACCTTGCTCCGAGAACCTTTCTGGCCCTGGCGGATCTGTGGACCCTGTG
ACAATGGACGAGGCCAGCGAGACAAGCAGCTGTCCTTCTGACCTGGCCAGCAAGCCTAGACCTGAGGGCACAAGCCC
TAGCAGCTTCGAGTACACCATTCTGGACCCCAGCAGCCAGCTGCTGTGTCCTAGAGCACTGCCTCCAGAGCTGCCTC
CTACACCTCCTCACCTGAAGTTTCTGTTTCTGGTGGTGTCCGACAGCGGCATCAGCACCGATTATAGCTCTGGTGGC
TCTCAGGGCGTGCACGGCGATAGTTCTGATGGCCCTTACTCTCACCCCTACGAAAACAGCCTGGTGCCTGACAGCGA
GCCTCTGCACCCTGGATATGTGGCCTGTAGCTGA
14. SEQ ID NO: 14 Mouse orthoIL2Rb-EpoR(ITIM YF) (moRb-EpoR(YF) protein sequence
(SEQ ID NO: 14)
MATIALPWSLSLYVFLLLLATPWASAAVKNCSHLECFYNSRANVSCMWSHEEALNVTTCHVHAKSNLRHWNKTCELT
LVRQASWACNLILGSFPESQSLTSVDLLDINVVCWEEKGWRRVKTCDFHPFDNLRLVAPHSLQVLHIDTQRCNISWK
VSQVSDFIEPYLEFEARRRLLGHSWEDASVLSLKQRQQWLFLEMLIPSTSYEVQVRVKAQRNNTGTWSPWSQPLTFR
TRPASDLDPLILTLSLILVLISLLLTVLALLSHRRTLQQKIWPGIPSPESEFEGLFTTHKGNFQLWLLQRDGCLWWS
PGSSFPEDPPAHLEVLSEPRWAVTQAGDPGADDEGPLLEPVGSEHAQDTYLVLDKWLLPRTPCSENLSGPGGSVDPV
TMDEASETSSCPSDLASKPRPEGTSPSSFEYTILDPSSQLLCPRALPPELPPTPPHLKFLFLVVSDSGISTDYSSGG
SQGVHGDSSDGPYSHPYENSLVPDSEPLHPGYVACS
Residues 1-235 of the moRb-EpoR(YF) chimeric orthogonal receptor (SEQ ID NO:
14) are derived from the the mouse ortho IL-2Rβ (SEQ ID NO. 2) and residues 236-
498 (underlined) of moRb-EpoR(YF (SEQ ID NO: 14) are derived from the mouse
EpoR with the two Phe (“F”) residue substitutions indicated in bold.
B. Ortho Rb (hoRb) Receptor Sequences
1. SEQ ID NO: 15 Human orthoIL2Rb (hoRb) coding sequence
(SEQ ID NO: 15)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAGCCCTTGGGAAGGACACCATTCCGTGGCTCGGCCACCTCCTCGTGGGTCTCAGCGGGGCTTTTGGCTT
CATCATCTTAGTGTACTTGCTGATCAACTGCAGGAACACCGGGCCATGGCTGAAGAAGGTCCTGAAGTGTAACACCC
CAGACCCCTCGAAGTTCTTTTCCCAGCTGAGCTCAGAGCATGGAGGAGACGTCCAGAAGTGGCTCTCTTCGCCCTTC
CCCTCATCGTCCTTCAGCCCTGGCGGCCTGGCACCTGAGATCTCGCCACTAGAAGTGCTGGAGAGGGACAAGGTGAC
GCAGCTGCTCCTGCAGCAGGACAAGGTGCCTGAGCCCGCATCCTTAAGCAGCAACCACTCGCTGACCAGCTGCTTCA
CCAACCAGGGTTACTTCTTCTTCCACCTCCCGGATGCCTTGGAGATAGAGGCCTGCCAGGTGTACTTTACTTACGAC
CCCTACTCAGAGGAAGACCCTGATGAGGGTGTGGCCGGGGCACCCACAGGGTCTTCCCCCCAACCCCTGCAGCCTCT
GTCAGGGGAGGACGACGCCTACTGCACCTTCCCCTCCAGGGATGACCTGCTGCTCTTCTCCCCCAGTCTCCTCGGTG
GCCCCAGCCCCCCAAGCACTGCCCCTGGGGGCAGTGGGGCCGGTGAAGAGAGGATGCCCCCTTCTTTGCAAGAAAGA
GTCCCCAGAGACTGGGACCCCCAGCCCCTGGGGCCTCCCACCCCAGGAGTCCCAGACCTGGTGGATTTTCAGCCACC
CCCTGAGCTGGTGCTGCGAGAGGCTGGGGAGGAGGTCCCTGACGCTGGCCCCAGGGAGGGAGTCAGTTTCCCCTGGT
CCAGGCCTCCTGGGCAGGGGGAGTTCAGGGCCCTTAATGCTCGCCTGCCCCTGAACACTGATGCCTACTTGTCCCTC
CAAGAACTCCAGGGTCAGGACCCAACTCACTTGGTGTAG
2. SEQ ID NO: 16 Human orthoIL2Rb (hoRb) protein sequence:
(SEQ ID NO: 16)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPAALGKDTIPWLGHLLVGLSGAFGFIILVYLLINCRNTGPWLKKVLKCNTPDPSKFFSQLSSEHGGDVQKWLSSPF
PSSSFSPGGLAPEISPLEVLERDKVTQLLLQQDKVPEPASLSSNHSLTSCFTNQGYFFFHLPDALEIEACQVYFTYD
PYSEEDPDEGVAGAPTGSSPQPLQPLSGEDDAYCTFPSRDDLLLFSPSLLGGPSPPSTAPGGSGAGEERMPPSLQER
VPRDWDPQPLGPPTPGVPDLVDFQPPPELVLREAGEEVPDAGPREGVSFPWSRPPGQGEFRALNARLPLNTDAYLSL
QELQGQDPTHLV
The hoRb orthogonal receptor protein (SEQ ID NO: 16) corresponds to the wildtype
human IL2Rβ (hCD122) protein but containing the amino acid substitutions H133D
and Y134F, relative to the wild type hCD122 protein.
3. SEQ ID NO: 17 human orthoIL2Rb-IL7 (hoRb-IL7R) coding sequence
(SEQ ID NO: 17)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAAATAATAGCTCAGGGGAGATGGATCCTATCTTACTAACCATCAGCATTTTGAGTTTTTTCTCTGTCGC
TCTGTTGGTCATCTTGGCCTGTGTGTTATGGAAAAAAAGGATTAAGCCTATCGTATGGCCCAGTCTCCCCGATCATA
AGAAGACTCTGGAACATCTTTGTAAGAAACCAAGAAAAAATTTAAATGTGAGTTTCAATCCTGAAAGTTTCCTGGAC
TGCCAGATTCATAGGGTGGATGACATTCAAGCTAGAGATGAAGTGGAAGGTTTTCTGCAAGATACGTTTCCTCAGCA
ACTAGAAGAATCTGAGAAGCAGAGGCTTGGAGGGGATGTGCAGAGCCCCAACTGCCCATCTGAGGATGTAGTCATCA
CTCCAGAAAGCTTTGGAAGAGATTCATCCCTCACATGCCTGGCTGGGAATGTCAGTGCATGTGACGCCCCTATTCTC
TCCTCTTCCAGGTCCCTAGACTGCAGGGAGAGTGGCAAGAATGGGCCTCATGTGTACCAGGACCTCCTGCTTAGCCT
TGGGACTACAAACAGCACGCTGCCCCCTCCATTTTCTCTCCAATCTGGAATCCTGACATTGAACCCAGTTGCTCAGG
GTCAGCCCATTCTTACTTCCCTGGGATCAAATCAAGAAGAAGCATATGTCACCATGTCCAGCTTCTACCAAAACCAG
TGA
4. SEQ ID NO: 18 Human orthoIL2Rb-IL7 (hoRb-IL7R) protein sequence:
(SEQ ID NO: 18)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPANNSSGEMDPILLTISILSFFSVALLVILACVLWKKRIKPIVWPSLPDHKKTLEHLCKKPRKNLNVSFNPESFLD
CQIHRVDDIQARDEVEGFLQDTFPQQLEESEKQRLGGDVQSPNCPSEDVVITPESFGRDSSLTCLAGNVSACDAPIL
SSSRSLDCRESGKNGPHVYQDLLLSLGTTNSTLPPPFSLQSGILTLNPVAQGQPILTSLGSNQEEAYVTMSSFYQNQ
Residues 1-234 of the hoRb-IL7R chimeric orthogonal receptor (SEQ ID NO: 18) are
derived from human ortho IL-2Rβ (SEQ ID NO: 16) and residues 235-462 (underlined)
of the hoRb-IL7 chimeric orthogonal receptor are derived from human IL-7R.
5. SEQ ID NO: 19 human orthoIL2Rb-IL7Rtail (hoRb-IL7Rtail) coding sequence:
(SEQ ID NO: 19)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAGCCCTTGGGAAGGACACCATTCCGTGGCTCGGCCACCTCCTCGTGGGTCTCAGCGGGGCTTTTGGCTT
CATCATCTTAGTGTACTTGCTGATCAACTGCAGGAACACCGGGCCATGGCTGAAGAAGGTCCTGAAGTGTAACACCC
CAGACCCCTCGAAGTTCTTTTCCCAGCTGAGCTCAGAGCATGGAGGAGACGTCCAGAAGTGGCTCTCTTCGCCCTTC
CCCTCATCGTCCTTCAGCCCTGGCGGCCTGGCACCTGAGATCTCGCCACTAGAAGTGCTGGAGAGGGACAAGGTGAC
GCAGCTGCTCCTGCAGCAGGACAAGGTGCCTGAGCCCGCATCCTTAAGCAGCAACCACTCGCTGACCAGCTGCTTCA
CCAACCAGGGTTACTTCTTCTTCCACCTCCCGGATGCCTTGGAGATAGAGGCCTGCCAGGTGTACTTTACTTACGAC
CCCTACTCAGAGGAAGACCCTGATGAGGGTGTGGCCGGGGCACCCACAGGGTCTTCCCCCCAACCCCTGCAGCCTCT
GTCAGGGGAGGACGACGCCTACTGCACCTTCCCCTCCAGGGATGACCTGCTGCTCTTCTCCCCCAGTCTCCTCGGTG
GCCCCAGCCCCCCAAGCACTGCCCCTGGGGGCAGTGGGGCCGGTGAAGAGAGGATGCCCCCTTCTTTGCAAGAAAGA
GTCCCCAGAGACTGGGACCCCCAGCCCCTGGGGCCTCCCACCCCAGGAGTCCCAGACCTGGTGGATTTTCAGCCACC
CCCTGAGCTGGTGCTGCGAGAGGCTGGGGAGGAGGTCCCTGACGCTGGCCCCAGGGAGGGAGTCAGTTTCCCCTGGT
CCAGGCCTCCTGGGCAGGGGGAGTTCAGGGCCCTTAATGCTCGCCTGCCCCTGAACCAAGAAGAAGCATATGTCACC
ATGTCCAGCTTCTACCAAAACCAGTGA
6. SEQ ID NO: 20 human orthoIL2Rb-IL7Rtail (hoRb-IL7Rtail) protein sequence:
(SEQ ID NO: 20)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPAALGKDTIPWLGHLLVGLSGAFGFIILVYLLINCRNTGPWLKKVLKCNTPDPSKFFSQLSSEHGGDVQKWLSSPF
PSSSFSPGGLAPEISPLEVLERDKVTQLLLQQDKVPEPASLSSNHSLTSCFTNQGYFFFHLPDALEIEACQVYFTYD
PYSEEDPDEGVAGAPTGSSPQPLQPLSGEDDAYCTFPSRDDLLLFSPSLLGGPSPPSTAPGGSGAGEERMPPSLQER
VPRDWDPQPLGPPTPGVPDLVDFQPPPELVLREAGEEVPDAGPREGVSFPWSRPPGQGEFRALNARLPLNQEEAYVT
MSSFYQNQ
Residues 1-532 of the hoRb-IL7Rtail chimeric orthogonal receptor (SEQ ID NO: 20)
are derived from human ortho IL-2Rβ (SEQ ID NO: 16) and residues 533-547
(underlined) of hoRb-IL7Rtail (SEQ ID NO: 20) are derived from human IL-7R.
7. SEQ ID NO: 21 human orthoIL2Rb-IL9R (hoRb-IL9R) coding sequence:
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCACAGAGACAAGGCCCTCTGATCCCACCCTGGGGGTGGCCAGGCAACACCCTTGTTGCTGTGTCCATCTT
TCTCCTGCTGACTGGCCCGACCTACCTCCTGTTCAAGCTGTCGCCCAGGGTGAAGAGAATCTTCTACCAGAACGTGC
CCTCTCCAGCGATGTTCTTCCAGCCCCTCTACAGTGTACACAATGGGAACTTCCAGACTTGGATGGGGGCCCACGGG
GCCGGTGTGCTGTTGAGCCAGGACTGTGCTGGCACCCCACAGGGAGCCTTGGAGCCCTGCGTCCAGGAGGCCACTGC
ACTGCTCACTTGTGGCCCAGCGCGTCCTTGGAAATCTGTGGCCCTGGAGGAGGAACAGGAGGGCCCTGGGACCAGGC
TCCCGGGGAACCTGAGCTCAGAGGATGTGCTGCCAGCAGGGTGTACGGAGTGGAGGGTACAGACGCTTGCCTATCTG
CCACAGGAGGACTGGGCCCCCACGTCCCTGACTAGGCCGGCTCCCCCAGACTCAGAGGGCAGCAGGAGCAGCAGCAG
CAGCAGCAGCAGCAACAACAACAACTACTGTGCCTTGGGCTGCTATGGGGGATGGCACCTCTCAGCCCTCCCAGGAA
ACACACAGAGCTCTGGGCCCATCCCAGCCCTGGCCTGTGGCCTTTCTTGTGACCATCAGGGCCTGGAGACCCAGCAA
GGAGTTGCCTGGGTGCTGGCTGGTCACTGCCAGAGGCCTGGGCTGCATGAGGACCTCCAGGGCATGTTGCTCCCTTC
TGTCCTCAGCAAGGCTCGGTCCTGGACATTCTA
8. SEQ ID NO: 22 human orthoIL2Rb-IL9R (hoRb-IL9R) protein sequence:
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPAQRQGPLIPPWGWPGNTLVAVSIFLLLTGPTYLLFKLSPRVKRIFYQNVPSPAMFFQPLYSVHNGNFQTWMGAHG
AGVLLSQDCAGTPQGALEPCVQEATALLTCGPARPWKSVALEEEQEGPGTRLPGNLSSEDVLPAGCTEWRVQTLAYL
PQEDWAPTSLTRPAPPDSEGSRSSSSSSSSNNNNYCALGCYGGWHLSALPGNTQSSGPIPALACGLSCDHQGLETQQ
GVAWVLAGHCQRPGLHEDLQGMLLPSVLSKARSWTF
Residues 1-234 of the chimeric orthogonal receptor hoRb-IL9R (SEQ ID NO: 22) are
derived from the human ortho IL-2Rβ (SEQ ID NO: 16)and residues 235-498
(underlined) of hoRb-IL9R are derived from the human IL-9R.
9. SEQ ID NO: 23 human orthoIL2Rb-IL21R (hoRb-IL21R) coding sequence
(SEQ ID NO: 23)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAGAGGAGTTAAAGGAAGGCTGGAACCCTCACCTGCTGCTTCTCCTCCTGCTTGTCATAGTCTTCATTCC
TGCCTTCTGGAGCCTGAAGACCCATCCATTGTGGAGGCTATGGAAGAAGATATGGGCCGTCCCCAGCCCTGAGCGGT
TCTTCATGCCCCTGTACAAGGGCTGCAGCGGAGACTTCAAGAAATGGGTGGGTGCACCCTTCACTGGCTCCAGCCTG
GAGCTGGGACCCTGGAGCCCAGAGGTGCCCTCCACCCTGGAGGTGTACAGCTGCCACCCACCACGGAGCCCGGCCAA
GAGGCTGCAGCTCACGGAGCTACAAGAACCAGCAGAGCTGGTGGAGTCTGACGGTGTGCCCAAGCCCAGCTTCTGGC
CGACAGCCCAGAACTCGGGGGGCTCAGCTTACAGTGAGGAGAGGGATCGGCCATACGGCCTGGTGTCCATTGACACA
GTGACTGTGCTAGATGCAGAGGGGCCATGCACCTGGCCCTGCAGCTGTGAGGATGACGGCTACCCAGCCCTGGACCT
GGATGCTGGCCTGGAGCCCAGCCCAGGCCTAGAGGACCCACTCTTGGATGCAGGGACCACAGTCCTGTCCTGTGGCT
GTGTCTCAGCTGGCAGCCCTGGGCTAGGAGGGCCCCTGGGAAGCCTCCTGGACAGACTAAAGCCACCCCTTGCAGAT
GGGGAGGACTGGGCTGGGGGACTGCCCTGGGGTGGCCGGTCACCTGGAGGGGTCTCAGAGAGTGAGGCGGGCTCACC
CCTGGCCGGCCTGGATATGGACACGTTTGACAGTGGCTTTGTGGGCTCTGACTGCAGCAGCCCTGTGGAGTGTGACT
TCACCAGCCCCGGGGACGAAGGACCCCCCCGGAGCTACCTCCGCCAGTGGGTGGTCATTCCTCCGCCACTTTCGAGC
CCTGGACCCCAGGCCAGCTAA
10. SEQ ID NO: 24 human orthoIL2Rb-IL21R (hoRb-IL21R) protein sequence:
(SEQ ID NO: 24)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPAEELKEGWNPHLLLLLLLVIVFIPAFWSLKTHPLWRLWKKIWAVPSPERFFMPLYKGCSGDFKKWVGAPFTGSSL
ELGPWSPEVPSTLEVYSCHPPRSPAKRLQLTELQEPAELVESDGVPKPSFWPTAQNSGGSAYSEERDRPYGLVSIDT
VTVLDAEGPCTWPCSCEDDGYPALDLDAGLEPSPGLEDPLLDAGTTVLSCGCVSAGSPGLGGPLGSLLDRLKPPLAD
GEDWAGGLPWGGRSPGGVSESEAGSPLAGLDMDTFDSGFVGSDCSSPVECDFTSPGDEGPPRSYLRQWVVIPPPLSS
PGPQAS
Residues 1-234 of the hoRb-IL21R chimeric orthogonal receptor (SEQ ID NO: 24)
are derived from the human ortho IL-2Rβ (SEQ ID NO: 16) and underlined residues
235-545 hoRb-IL21R are derived from the human IL-21R
11. SEQ ID NO: 25 human orthoIL2Rb-EpoR (hoRb-EpoR) coding sequence
(SEQ ID NO: 25)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAAGCGACCTGGACCCCCTCATCCTGACGCTCTCCCTCATCCTCGTGGTCATCCTGGTGCTGCTGACCGT
GCTCGCGCTGCTCTCCCACCGCCGGGCTCTGAAGCAGAAGATCTGGCCTGGCATCCCGAGCCCAGAGAGCGAGTTTG
AAGGCCTCTTCACCACCCACAAGGGTAACTTCCAGCTGTGGCTGTACCAGAATGATGGCTGCCTGTGGTGGAGCCCC
TGCACCCCCTTCACGGAGGACCCACCTGCTTCCCTGGAAGTCCTCTCAGAGCGCTGCTGGGGGACGATGCAGGCAGT
GGAGCCGGGGACAGATGATGAGGGCCCCCTGCTGGAGCCAGTGGGCAGTGAGCATGCCCAGGATACCTATCTGGTGC
TGGACAAATGGTTGCTGCCCCGGAACCCGCCCAGTGAGGACCTCCCAGGGCCTGGTGGCAGTGTGGACATAGTGGCC
ATGGATGAAGGCTCAGAAGCATCCTCCTGCTCATCTGCTTTGGCCTCGAAGCCCAGCCCAGAGGGAGCCTCTGCTGC
CAGCTTTGAGTACACTATCCTGGACCCCAGCTCCCAGCTCTTGCGTCCATGGACACTGTGCCCTGAGCTGCCCCCTA
CCCCACCCCACCTAAAGTACCTGTACCTTGTGGTATCTGACTCTGGCATCTCAACTGACTACAGCTCAGGGGACTCC
CAGGGAGCCCAAGGGGGCTTATCCGATGGCCCCTACTCCAACCCTTATGAGAACAGCCTTATCCCAGCCGCTGAGCC
TCTGCCCCCCAGCTATGTGGCTTGCTCTTAG
12. SEQ ID NO: 26 human orthoIL2Rb-EpoR (hoRb-EpoR) protein sequence
(SEQ ID NO: 26)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPASDLDPLILTLSLILVVILVLLTVLALLSHRRALKQKIWPGIPSPESEFEGLFTTHKGNFQLWLYQNDGCLWWSP
CTPFTEDPPASLEVLSERCWGTMQAVEPGTDDEGPLLEPVGSEHAQDTYLVLDKWLLPRNPPSEDLPGPGGSVDIVA
MDEGSEASSCSSALASKPSPEGASAASFEYTILDPSSQLLRPWTLCPELPPTPPHLKYLYLVVSDSGISTDYSSGDS
QGAQGGLSDGPYSNPYENSLIPAAEPLPPSYVACS
Residues 1-234 of the hoRb-EpoR chimeric orthogonal receptor (SEQ ID NO: 26) are
derived from the human ortho IL-2Rβ (SEQ ID NO: 16) and residues 235-497
(underlined) hoRb-EpoR are derived from human EpoR.
13. SEQ ID NO: 27 human orthoIL2Rb-EpoR(ITIM YF) (hoRb-EpoR(YF) coding sequence:
(SEQ ID NO: 27)
ATGGCGGCCCCTGCTCTGTCCTGGCGTCTGCCCCTCCTCATCCTCCTCCTGCCCCTGGCTACCTCTTGGGCATCTGC
AGCGGTGAATGGCACTTCCCAGTTCACATGCTTCTACAACTCGAGAGCCAACATCTCCTGTGTCTGGAGCCAAGATG
GGGCTCTGCAGGACACTTCCTGCCAAGTCCATGCCTGGCCGGACAGACGGCGGTGGAACCAAACCTGTGAGCTGCTC
CCCGTGAGTCAAGCATCCTGGGCCTGCAACCTGATCCTCGGAGCCCCAGATTCTCAGAAACTGACCACAGTTGACAT
CGTCACCCTGAGGGTGCTGTGCCGTGAGGGGGTGCGATGGAGGGTGATGGCCATCCAGGACTTCAAGCCCTTTGAGA
ACCTTCGCCTGATGGCCCCCATCTCCCTCCAAGTTGTCCACGTGGAGACCCACAGATGCAACATAAGCTGGGAAATC
TCCCAAGCCTCCgACTtCTTTGAAAGACACCTGGAGTTCGAGGCCCGGACGCTGTCCCCAGGCCACACCTGGGAGGA
GGCCCCCCTGCTGACTCTCAAGCAGAAGCAGGAATGGATCTGCCTGGAGACGCTCACCCCAGACACCCAGTATGAGT
TTCAGGTGCGGGTCAAGCCTCTGCAAGGCGAGTTCACGACCTGGAGCCCCTGGAGCCAGCCCCTGGCCTTCAGGACA
AAGCCTGCAAGCGACCTGGACCCCCTCATCCTGACGCTCTCCCTCATCCTCGTGGTCATCCTGGTGCTGCTGACCGT
GCTCGCGCTGCTCTCCCACCGCCGGGCTCTGAAGCAGAAGATCTGGCCTGGCATCCCGAGCCCAGAGAGCGAGTTTG
AAGGCCTCTTCACCACCCACAAGGGTAACTTCCAGCTGTGGCTGTACCAGAATGATGGCTGCCTGTGGTGGAGCCCC
TGCACCCCCTTCACGGAGGACCCACCTGCTTCCCTGGAAGTCCTCTCAGAGCGCTGCTGGGGGACGATGCAGGCAGT
GGAGCCGGGGACAGATGATGAGGGCCCCCTGCTGGAGCCAGTGGGCAGTGAGCATGCCCAGGATACCTATCTGGTGC
TGGACAAATGGTTGCTGCCCCGGAACCCGCCCAGTGAGGACCTCCCAGGGCCTGGTGGCAGTGTGGACATAGTGGCC
ATGGATGAAGGCTCAGAAGCATCCTCCTGCTCATCTGCTTTGGCCTCGAAGCCCAGCCCAGAGGGAGCCTCTGCTGC
CAGCTTTGAGTACACTATCCTGGACCCCAGCTCCCAGCTCTTGCGTCCATGGACACTGTGCCCTGAGCTGCCCCCTA
CCCCACCCCACCTAAAGTTCCTGTTCCTTGTGGTATCTGACTCTGGCATCTCAACTGACTACAGCTCAGGGGACTCC
CAGGGAGCCCAAGGGGGCTTATCCGATGGCCCCTACTCCAACCCTTATGAGAACAGCCTTATCCCAGCCGCTGAGCC
TCTGCCCCCCAGCTATGTGGCTTGCTCTTAG
14. SEQ ID NO: 28 human orthoIL2Rb-EpoR(ITIM YF) (hoRb-EpoR(YF)) protein sequence
(SEQ ID NO: 28)
MAAPALSWRLPLLILLLPLATSWASAAVNGTSQFTCFYNSRANISCVWSQDGALQDTSCQVHAWPDRRRWNQTCELL
PVSQASWACNLILGAPDSQKLTTVDIVTLRVLCREGVRWRVMAIQDFKPFENLRLMAPISLQVVHVETHRCNISWEI
SQASDFFERHLEFEARTLSPGHTWEEAPLLTLKQKQEWICLETLTPDTQYEFQVRVKPLQGEFTTWSPWSQPLAFRT
KPASDLDPLILTLSLILVVILVLLTVLALLSHRRALKQKIWPGIPSPESEFEGLFTTHKGNFQLWLYQNDGCLWWSP
CTPFTEDPPASLEVLSERCWGTMQAVEPGTDDEGPLLEPVGSEHAQDTYLVLDKWLLPRNPPSEDLPGPGGSVDIVA
MDEGSEASSCSSALASKPSPEGASAASFEYTILDPSSQLLRPWTLCPELPPTPPHLKFLFLVVSDSGISTDYSSGDS
QGAQGGLSDGPYSNPYENSLIPAAEPLPPSYVACS
Residues 1-234 of the hoRb-EpoR(YF) chimeric orthogonal receptor (SEQ ID NO: 28)
are derived from the human ortho IL-2Rβ (SEQ ID NO: 16) and residues 235-497
(underlined) hoRb-EpoR are derived from human EpoR with the substituted residues
indicated in bold.
C. Orthogonal Ligands
1. SEQ ID NO: 29 Mouse orthoIL2 (3A10) (moIL2) coding sequence
(SEQ ID NO: 29)
ATGTACAGCATGCAGCTCGCATCCTGTGTCACATTGACACTTGTGCTCCTTGTCAACAGCGCACCCACTTCAAGCTC
CACTTCAAGCTCTACAGCGGAAGCACAGCAGCAGCAGCAGCAGCAGCAGCAGCAGCAGCAGCACCTGGACAACCTGT
TGGTGCTGCTAAAGGCCCTCCTGAGCAGGATGGAGAATTACAGGAACCTGAAACTCCCCAGGATGCTCACCTTCAAA
TTTTACTTGCCCAAGCAGGCCACAGAATTGAAAGATCTTCAGTGCCTAGAAGATGAACTTGGACCTCTGCGGCATGT
TCTGGATTTGACTCAAAGCAAAAGCTTTCAATTGGAAGATGCTGAGAATTTCATCAGCAATATCAGAGTAACTGTTG
TAAAACTAAAGGGCTCTGACAACACATTTGAGTGCCAATTCGATGATGAGTCAGCAACTGTGGTGGACTTTCTGAGG
AGATGGATAGCCTTCTGTCAAAGCATCATCTCAACAAGCCCTCAATAA
2. SEQ ID NO: 30 Mouse orthoIL2 (3A10) (moIL2) coding sequence:
(SEQ ID NO: 24)
MYSMQLASCVTLTLVLLVNSAPTSSSTSSSTAEAQQQQQQQQQQQQHLDNLLVLLKALLSRMENYRNLKLPRMLTFK
FYLPKQATELKDLQCLEDELGPLRHVLDLTQSKSFQLEDAENFISNIRVTVVKLKGSDNTFECQFDDESATVVDFLR
RWIAFCQSIISTSPQ
3A10/moIL2 (SEQ ID NO: 30) is a variant of murine IL2 containg the [E29D, Q30N,
M33V, D34L, Q36K, E37A] amino acid substitutions relative to wild type murine
IL2.
3. SEQ ID NO: 31 MSA-mouse orthoIL2(3A10)-6xHis (MSA-moIL2) coding sequence
(SEQ ID NO: 31)
ATGCTACTAGTAAATCAGTCACACCAAGGCTTCAATAAGGAACACACAAGCAAGATGGTAAGCGCTATTGTTTTATA
TGTGCTTTTGGCGGCGGCGGCGCATTCTGCCTTTGCGGGATCCAGGGGTGTGTTTCGCCGAGAAGCACACAAGAGTG
AGATCGCCCATCGGTATAATGATTTGGGAGAACAACATTTCAAAGGCCTAGTCCTGATTGCCTTTTCCCAGTATCTC
CAGAAATGCTCATACGATGAGCATGCCAAATTAGTGCAGGAAGTAACAGACTTTGCAAAGACGTGTGTTGCCGATGA
GTCTGCCGCCAACTGTGACAAATCCCTTCACACTCTTTTTGGAGATAAGTTGTGTGCCATTCCAAACCTCCGTGAAA
ACTATGGTGAACTGGCTGACTGCTGTACAAAACAAGAGCCCGAAAGAAACGAATGTTTCCTGCAACACAAAGATGAC
AACCCCAGCCTGCCACCATTTGAAAGGCCAGAGGCTGAGGCCATGTGCACCTCCTTTAAGGAAAACCCAACCACCTT
TATGGGACACTATTTGCATGAAGTTGCCAGAAGACATCCTTATTTCTATGCCCCAGAACTTCTTTACTATGCTGAGC
AGTACAATGAGATTCTGACCCAGTGTTGTGCAGAGGCTGACAAGGAAAGCTGCCTGACCCCGAAGCTTGATGGTGTG
AAGGAGAAAGCATTGGTCTCATCTGTCCGTCAGAGAATGAAGTGCTCCAGTATGCAGAAGTTTGGAGAGAGAGCTTT
TAAAGCATGGGCAGTAGCTCGTCTGAGCCAGACATTCCCCAATGCTGACTTTGCAGAAATCACCAAATTGGCAACAG
ACCTGACCAAAGTCAACAAGGAGTGCTGCCATGGTGACCTGCTGGAATGCGCAGATGACAGGGCGGAACTTGCCAAG
TACATGTGTGAAAACCAGGCGACTATCTCCAGCAAACTGCAGACTTGCTGCGATAAACCACTGTTGAAGAAAGCCCA
CTGTCTTAGTGAGGTGGAGCATGACACCATGCCTGCTGATCTGCCTGCCATTGCTGCTGATTTTGTTGAGGACCAGG
AAGTGTGCAAGAACTATGCTGAGGCCAAGGATGTCTTCCTGGGCACGTTCTTGTATGAATATTCAAGAAGACACCCT
GATTACTCTGTATCCCTGTTGCTGAGACTTGCTAAGAAATATGAAGCCACTCTGGAAAAGTGCTGCGCTGAAGCCAA
TCCTCCCGCATGCTACGGCACAGTGCTTGCTGAATTTCAGCCTCTTGTAGAAGAGCCTAAGAACTTGGTCAAAACCA
ACTGTGATCTTTACGAGAAGCTTGGAGAATATGGATTCCAAAATGCCATTCTAGTTCGCTACACCCAGAAAGCACCT
CAGGTGTCAACCCCAACTCTCGTGGAGGCTGCAAGAAACCTAGGAAGAGTGGGCACCAAGTGTTGTACACTTCCTGA
AGATCAGAGACTGCCTTGTGTGGAAGACTATCTGTCTGCAATCCTGAACCGTGTGTGTCTGCTGCATGAGAAGACCC
CAGTGAGTGAGCATGTTACCAAGTGCTGTAGTGGATCCCTGGTGGAAAGGCGGCCATGCTTCTCTGCTCTGACAGTT
GATGAAACATATGTCCCCAAAGAGTTTAAAGCTGAGACCTTCACCTTCCACTCTGATATCTGCACACTTCCAGAGAA
GGAGAAGCAGATTAAGAAACAAACGGCTCTTGCTGAGCTGGTGAAGCACAAGCCCAAGGCTACAGCGGAGCAACTGA
AGACTGTCATGGATGACTTTGCACAGTTCCTGGATACATGTTGCAAGGCTGCTGACAAGGACACCTGCTTCTCGACT
GAGGGTCCAAACCTTGTCACTAGATGCAAAGACGCCTTAGCCGGCGGTGGCGGTTCAGCACCCACTTCAAGCTCCAC
TTCAAGCTCTACAGCGGAAGCACAGCAGCAGCAGCAGCAGCAGCAGCAGCAGCAGCAGCACCTGGATAATCTGTTGG
TGCTGCTAAAGGCGCTCCTGAGCAGGATGGAGAATTACAGGAACCTGAAACTCCCCAGGATGCTCACCTTCAAATTT
TACTTGCCCAAGCAGGCCACAGAATTGAAAGATCTTCAGTGCCTAGAAGATGAACTTGGACCTCTGCGGCATGTTCT
GGATTTGACTCAAAGCAAAAGCTTTCAATTGGAAGATGCTGAGAATTTCATCAGCAATATCAGAGTAACTGTTGTAA
AACTAAAGGGCTCTGACAACACATTTGAGTGCCAATTCGATGATGAGTCAGCAACTGTGGTGGACTTTCTGAGGAGA
TGGATAGCCTTCTGTCAAAGCATCATCTCAACAAGCCCTCAAGCGGCCGCGCATCATCACCACCATCACCACCATTA
A
4. SEQ ID NO: 32 MSA-mouse orthoIL2(3A10)-6xHis (MSA-moIL2) protein sequence
(SEQ ID NO: 32)
MLLVNQSHQGFNKEHTSKMVSAIVLYVLLAAAAHSAFAGSRGVFRREAHKSEIAHRYNDLGEQHFKGLVLIAFSQYL
QKCSYDEHAKLVQEVTDFAKTCVADESAANCDKSLHTLFGDKLCAIPNLRENYGELADCCTKQEPERNECFLQHKDD
NPSLPPFERPEAEAMCTSFKENPTTFMGHYLHEVARRHPYFYAPELLYYAEQYNEILTQCCAEADKESCLTPKLDGV
KEKALVSSVRQRMKCSSMQKFGERAFKAWAVARLSQTFPNADFAEITKLATDLTKVNKECCHGDLLECADDRAELAK
YMCENQATISSKLQTCCDKPLLKKAHCLSEVEHDTMPADLPAIAADFVEDQEVCKNYAEAKDVFLGTFLYEYSRRHP
DYSVSLLLRLAKKYEATLEKCCAEANPPACYGTVLAEFQPLVEEPKNLVKTNCDLYEKLGEYGFQNAILVRYTQKAP
QVSTPTLVEAARNLGRVGTKCCTLPEDQRLPCVEDYLSAILNRVCLLHEKTPVSEHVTKCCSGSLVERRPCFSALTV
DETYVPKEFKAETFTFHSDICTLPEKEKQIKKQTALAELVKHKPKATAEQLKTVMDDFAQFLDTCCKAADKDTCFST
EGPNLVTRCKDALAGGGGSAPTSSSTSSSTAEAQQQQQQQQQQQQHLDNLLVLLKALLSRMENYRNLKLPRMLTFKF
YLPKQATELKDLQCLEDELGPLRHVLDLTQSKSFQLEDAENFISNIRVTVVKLKGSDNTFECQFDDESATVVDFLRR
WIAFCQSIISTSPQAAAHHHHHHHH
The MSA-moIL2 protein (SEQ ID NO: 32) is a variant of murine IL2 that contains
the following amino acid substitutions relative to the wild type mouse IL2:
[E29D, Q30N, M33V, D34L, Q36K, E37A] and and provides the addition of Ala-
Ala-Ala-His₆ polypeptide tag to the C-terminus of the human IL2 sequence
(underlined).
5. SEQ ID NO: 33 Human orthoIL2 (SQVLKA) (hoIL2) coding sequence
(SEQ ID NO: 33)
ATGTATAGGATGCAGTTGCTCAGTTGTATAGCACTGTCCCTTGCGCTGGTTACGAACAGCGCACCTACTTCAAGTTC
TACAAAGAAAACACAGCTACAACTGagccaaTTACTTgTGctgTTAaAGgcGATTTTGAATGGAATTAATAATTACA
AGAATCCCAAACTCACCAGGATGCTCACATTTAAGTTTTACATGCCCAAGAAGGCCACAGAACTGAAACATCTTCAG
TGTCTAGAAGAAGAACTCAAACCTCTGGAGGAAGTGCTAAATTTAGCTCAAAGCAAAAACTTTCACTTAAGACCCAG
GGACTTAATCAGCAATATCAACGTAATAGTTCTGGAACTAAAGGGATCTGAAACAACATTCATGTGTGAATATGCTG
ATGAGACAGCAACCATTGTAGAATTTCTGAACAGATGGATTACCTTTTGTCAAAGCATCATCTCAACACTAACTGCG
GCCGCCCACCATCACCATCACCATTAG
6. SEQ ID NO: 34 Human orthoIL2 (SQVLKA) (hoIL2) protein sequence
(SEQ ID NO: 34)
MYRMQLLSCIALSLALVTNS [symbol] APTSSSTKKTQLQLSQLLVLLKAILNGINNYKNPKLTRMLTFKFYMPKKATELKH
LQCLEEELKPLEEVLNLAQSKNFHLRPRDLISNINVIVLELKGSETTFMCEYADETATIVEFLNRWITFCQSIISTL
T
SQVLKA (SEQ ID NO: 34) is a variant derived from human IL2 containing the amino
acid substitutions [E15S, H16Q, L19V, D20L, Q22K, M23A] relative to wild-
type human IL2.
7. SEQ ID NO: 35 MSA-Human orthoIL2-6xHis (MSA-hoIL2) (baculovirus vector)
coding sequence
(SEQ ID NO: 35)
ATGCTACTAGTAAATCAGTCACACCAAGGCTTCAATAAGGAACACACAAGCAAGATGGTAAGCGCTATTGTTTTATA
TGTGCTTTTGGCGGCGGCGGCGCATTCTGCCTTTGCGGGATCCAGGGGTGTGTTTCGCCGAGAAGCACACAAGAGTG
AGATCGCCCATCGGTATAATGATTTGGGAGAACAACATTTCAAAGGCCTAGTCCTGATTGCCTTTTCCCAGTATCTC
CAGAAATGCTCATACGATGAGCATGCCAAATTAGTGCAGGAAGTAACAGACTTTGCAAAGACGTGTGTTGCCGATGA
GTCTGCCGCCAACTGTGACAAATCCCTTCACACTCTTTTTGGAGATAAGTTGTGTGCCATTCCAAACCTCCGTGAAA
ACTATGGTGAACTGGCTGACTGCTGTACAAAACAAGAGCCCGAAAGAAACGAATGTTTCCTGCAACACAAAGATGAC
AACCCCAGCCTGCCACCATTTGAAAGGCCAGAGGCTGAGGCCATGTGCACCTCCTTTAAGGAAAACCCAACCACCTT
TATGGGACACTATTTGCATGAAGTTGCCAGAAGACATCCTTATTTCTATGCCCCAGAACTTCTTTACTATGCTGAGC
AGTACAATGAGATTCTGACCCAGTGTTGTGCAGAGGCTGACAAGGAAAGCTGCCTGACCCCGAAGCTTGATGGTGTG
AAGGAGAAAGCATTGGTCTCATCTGTCCGTCAGAGAATGAAGTGCTCCAGTATGCAGAAGTTTGGAGAGAGAGCTTT
TAAAGCATGGGCAGTAGCTCGTCTGAGCCAGACATTCCCCAATGCTGACTTTGCAGAAATCACCAAATTGGCAACAG
ACCTGACCAAAGTCAACAAGGAGTGCTGCCATGGTGACCTGCTGGAATGCGCAGATGACAGGGCGGAACTTGCCAAG
TACATGTGTGAAAACCAGGCGACTATCTCCAGCAAACTGCAGACTTGCTGCGATAAACCACTGTTGAAGAAAGCCCA
CTGTCTTAGTGAGGTGGAGCATGACACCATGCCTGCTGATCTGCCTGCCATTGCTGCTGATTTTGTTGAGGACCAGG
AAGTGTGCAAGAACTATGCTGAGGCCAAGGATGTCTTCCTGGGCACGTTCTTGTATGAATATTCAAGAAGACACCCT
GATTACTCTGTATCCCTGTTGCTGAGACTTGCTAAGAAATATGAAGCCACTCTGGAAAAGTGCTGCGCTGAAGCCAA
TCCTCCCGCATGCTACGGCACAGTGCTTGCTGAATTTCAGCCTCTTGTAGAAGAGCCTAAGAACTTGGTCAAAACCA
ACTGTGATCTTTACGAGAAGCTTGGAGAATATGGATTCCAAAATGCCATTCTAGTTCGCTACACCCAGAAAGCACCT
CAGGTGTCAACCCCAACTCTCGTGGAGGCTGCAAGAAACCTAGGAAGAGTGGGCACCAAGTGTTGTACACTTCCTGA
AGATCAGAGACTGCCTTGTGTGGAAGACTATCTGTCTGCAATCCTGAACCGTGTGTGTCTGCTGCATGAGAAGACCC
CAGTGAGTGAGCATGTTACCAAGTGCTGTAGTGGATCCCTGGTGGAAAGGCGGCCATGCTTCTCTGCTCTGACAGTT
GATGAAACATATGTCCCCAAAGAGTTTAAAGCTGAGACCTTCACCTTCCACTCTGATATCTGCACACTTCCAGAGAA
GGAGAAGCAGATTAAGAAACAAACGGCTCTTGCTGAGCTGGTGAAGCACAAGCCCAAGGCTACAGCGGAGCAACTGA
AGACTGTCATGGATGACTTTGCACAGTTCCTGGATACATGTTGCAAGGCTGCTGACAAGGACACCTGCTTCTCGACT
GAGGGTCCAAACCTTGTCACTAGATGCAAAGACGCCTTAGCCGGCGGTGGCGGTTCAcccgggGCACCTACTTCAAG
TTCTACAAAGAAAACACAGCTACAACTGagcCAaTTACTTgTGctgTTAaAGgcGATTTTGAATGGAATTAATAATT
ACAAGAATCCCAAACTCACCAGGATGCTCACATTTAAGTTTTACATGCCCAAGAAGGCCACAGAACTGAAACATCTT
CAGTGTCTAGAAGAAGAACTCAAACCTCTGGAGGAAGTGCTAAATTTAGCTCAAAGCAAAAACTTTCACTTAAGACC
CAGGGACTTAATCAGCAATATCAACGTAATAGTTCTGGAACTAAAGGGATCTGAAACAACATTCATGTGTGAATATG
CTGATGAGACAGCAACCATTGTAGAATTTCTGAACAGATGGATTACCTTTTGTCAAAGCATCATCTCAACACTAACT
GCGGCCGCGCATCATCACCACCATCACCACCATTAA
8. SEQ ID NO: 36 MSA-Human orthoIL2-6xHis (MSA-hoIL2) protein sequence
(SEQ ID NO: 36)
MLLVNQSHQGFNKEHTSKMVSAIVLYVLLAAAAHSAFAGSRGVFRREAHKSEIAHRYNDLGEQHFKGLVLIAFSQYL
QKCSYDEHAKLVQEVTDFAKTCVADESAANCDKSLHTLFGDKLCAIPNLRENYGELADCCTKQEPERNECFLQHKDD
NPSLPPFERPEAEAMCTSFKENPTTFMGHYLHEVARRHPYFYAPELLYYAEQYNEILTQCCAEADKESCLTPKLDGV
KEKALVSSVRQRMKCSSMQKFGERAFKAWAVARLSQTFPNADFAEITKLATDLTKVNKECCHGDLLECADDRAELAK
YMCENQATISSKLQTCCDKPLLKKAHCLSEVEHDTMPADLPAIAADFVEDQEVCKNYAEAKDVFLGTFLYEYSRRHP
DYSVSLLLRLAKKYEATLEKCCAEANPPACYGTVLAEFQPLVEEPKNLVKTNCDLYEKLGEYGFQNAILVRYTQKAP
QVSTPTLVEAARNLGRVGTKCCTLPEDQRLPCVEDYLSAILNRVCLLHEKTPVSEHVTKCCSGSLVERRPCFSALTV
DETYVPKEFKAETFTFHSDICTLPEKEKQIKKQTALAELVKHKPKATAEQLKTVMDDFAQFLDTCCKAADKDTCFST
EGPNLVTRCKDALAGGGGSPGAPTSSSTKKTQLQLSQLLVLLKAILNGINNYKNPKLTRMLTFKFYMPKKATELKHL
QCLEEELKPLEEVLNLAQSKNFHLRPRDLISNINVIVLELKGSETTFMCEYADETATIVEFLNRWITFCQSIISTLT
AAAHHHHHHHH
MSA-hoIL2 (SEQ ID NO: 36) is a polypeptide derived from wild-type human IL2 and
contains the following amino acid substitutions relative to wild type human
IL2: [E15S, H16Q, L19V, D20L, Q22K, M23A] and provides the addition of Ala-
Ala-Ala-His₆ polypeptide tag to the C-terminus of the human IL2 sequence
(underlined).

What is claimed is:

1. An orthogonal chimeric receptor polypeptide, comprising:
(a) an orthogonal ligand binding domain (oLBD) of an orthogonal receptor which (i) has significantly reduced binding to its native ligand; and which (ii) comprises at least one amino acid substitution relative to the sequence of the native protein;
(b) an intracellular domain (ICD) of a second receptor which binds to one or more JAK/STAT proteins and is not the orthogonal receptor; and
(c) a transmembrane domain (TMD) that operably joins the oLBD and the ICD.

2. The orthogonal chimeric receptor of claim 1, wherein both the TMD and the ICD are derived from the second receptor.

3. The orthogonal chimeric receptor polypeptide of claim 1 or claim 2, wherein the second receptor is a cytokine receptor.

4. The orthogonal chimeric receptor polypeptide of claim 3, wherein the second receptor is selected from CD121α; CDw121β; IL-18Rα; IL-18β; CD122; CD25; CD124; CD213; CD127; IL-9R; CD21α1; CD213α2; IL-15Rα; CD131; CD125; CD131; CD126; CD130; IL-11Rα; CD114; CD212; LIFR; OSMR; CD210; IL-20Rα, IL-20β; IL-14R; CD4; CD217; CD118; CD119; CD40; LTβR; CD120α; CD120β; CD137 (4-1BB); BCMA, TACI; CD27; CD30; CD95 (Fas); GITR; LTβR; HVEM; OX40; BCMA, TACI; TRAILR1-4; Apo3; RANK, OPG; TGF-βR1; TGF-βR2; TGF-βR3; EpoR; TpoR; Flt-3; CD117; CD115; CDw136;

5. The orthogonal chimeric receptor polypeptide of claim 3 wherein the second receptor is a receptor associated with the common gamma chain (CD132).

6. The orthogonal chimeric receptor polypeptide of claim 5 wherein the second receptor is selected from IL-4 receptor (IL-4R), IL-7 receptor (IL-7R), IL-9 receptor (IL-9R), IL-15Rα, IL-21 receptor (IL-21 Rα).

7. The orthogonal chimeric receptor polypeptide of claim 3 wherein the second receptor is the erythropoietin receptor (EpoR).

8. The orthogonal chimeric receptor polypeptide of any of claims 1-7, wherein the oLBD is an orthogonal variant of the CD122 ligand-binding domain.

9. The orthogonal chimeric receptor polypeptide of claim 8 wherein the CD122 receptor is human CD122 modified at one or more residues selected from R41, R42, Q70, K71, T73, T74, V75, S132, H133, Y134, F135, E136, Q214.

10. The orthogonal chimeric receptor polypeptide of claim 9 wherein the CD122 receptor comprises amino acid substitutions at H133 and Y134.

11. The orthogonal chimeric receptor polypeptide of claim 8 wherein the CD122 receptor is CD122 modified at one or more residues selected from R42, F67, Q71, S72, T74, S75, V76, S133, H134, Y135, 1136, E137, R215.

12. The orthogonal chimeric receptor polypeptide of claim 1, comprising an amino acid sequence with at least 75% sequence identity to any of SEQ ID NO:4, 6, 8, 10, 12, 14, 18, 20, 22, 24, 26, 28.

13. The orthogonal chimeric receptor polypeptide of claim 1, comprising an amino acid sequence with at least 95% sequence identity to any of SEQ ID NO:4, 6, 8, 10, 12, 14, 18, 20, 22, 24, 26, 28.

14. A system for selective activation of a receptor in a cell, the system comprising:
(a) an orthogonal chimeric receptor according to any of claims 1-13; and
(b) an engineered orthogonal ligand.

15. The system of claim 14, wherein the orthogonal chimeric receptor is expressed by a mammalian cell.

16. The system of claim 15, wherein the cell is an immune cell or a stem cell.

17. The system of claim 16, wherein the immune cell is a T cell.

18. The system of claim 17, wherein the T cell is a CAR T cell.

19. The system of any of claims 14-18, wherein the orthogonal ligand is IL-2.

20. The system of claim 19, wherein the orthologous IL-2 is human IL-2 modified at one or more residues selected from Q13, L14, E15, H16, L19, D20, Q22, M23, G27, and N88.

21. The system of claim 19, wherein the human IL-2 is modified at one or more residues selected from E15, H16, L19, D20, Q22, and M23.

22. The system of claim 19, wherein the orthologous IL-2 is mouse IL-2, modified at one or more residues selected from H27, L28, E29, Q30, M33, D34, Q36, E37, R41, and N103.

23. The system of claim 19, wherein the mouse IL-2 is modified at one or more residues selected from E29, Q30, M33, D34, Q36, and E37.

24. A nucleic acid encoding the orthologous chimeric receptor of any of claims 1-13.

25. An expression vector comprising the nucleic acid of claim 24.

26. A cell genetically engineered to comprise the vector of claim 25.

27. A method of treating an individual, the method comprising introducing an immune effector cell expressing an orthogonal chimeric receptor according to any of claim 1-13 and selectively activating the cell by contacting with an orthologous ligand.

28. The method of claim 27, wherein the immune effector cell is a T cell.

29. The method of claim 28, wherein the T cell is a CAR T cell.

30. The method of any of claims 27-29, wherein the individual is treated for cancer.

31. The method of any of claims 27-29, wherein the individual is treated for autoimmune disease.

32. The method of any of claims 27-29, wherein the individual is treated for infection.

33. A kit comprising the system of claim 14.
